(19) United States
(12) Patent Application Publication
(10) Pub. No.: US 2025/0195215 A1
(43) Pub. Date: Jun. 19, 2025

(54) PROSTHETIC VALVES WITH NON-UNIFORM VALVULAR STRUCTURES

(71) Applicant: EDWARDS LIFESCIENCES CORPORATION, Irvine, CA (US)

(72) Inventors: Michael Bukin, Pardes Hanna (IL); Noam Nir, Pardes-Hanna (IL); Eran Goldberg, Nesher (IL); Noa Axelrod Manela, Kadima (IL)

(73) Assignee: EDWARDS LIFESCIENCES CORPORATION, Irvine, CA (US)

(21) Appl. No.: 19/064,524

(22) Filed: Feb. 26, 2025

Related U.S. Application Data
(63) Continuation of application No. PCT/US2023/031756, filed on Aug. 31, 2023.
(60) Provisional application No. 63/403,484, filed on Sep. 2, 2022.

(51) Int. Cl. A61F 2/24

(52) U.S. Cl. CPC A61F 2/2418; A61F 2/2415; A61F 2220/0075; A61F 2240/001; A61F 2250/0018; A61F 2250/0037

(57) ABSTRACT

The present disclosure relates to prosthetic valves, and in particular, to prosthetic heart valves that include non-uniform valvular structures coupled to the frames of such valves, wherein the non-uniform valvular structures include soft or malleable movable portions configured to freely transition between closed and open configurations, and stiffer portions exhibiting increased stiffness or rigidity relative to the movable portions.

### CROSS-REFERENCE TO RELATED APPLICATIONS

This application is a continuation of PCT Application No. PCT/US2023/031756, filed on Aug. 31, 2023, which claims the benefit of U.S. Provisional Application No. 63/403,484, filed on Sep. 2, 2022, each of which is incorporated by reference herein in its entirety.

### FIELD

The present disclosure relates to prosthetic valves, and in particular, to prosthetic heart valves that include non-uniform valvular structures coupled to the frames of such valves, wherein the non-uniform valvular structures include soft or malleable movable portions configured to freely transition between closed and open configurations, and stiffer portions exhibiting increased stiffness or rigidity relative to the movable portions.

### BACKGROUND

Native heart valves, such as the aortic, pulmonary and mitral valves, function to assure adequate directional flow from, and to, the heart, and between the heart's chambers, to supply blood to the whole cardiovascular system. Various valvular diseases can render the valves ineffective and require replacement with artificial valves. Surgical procedures can be performed to repair or replace a heart valve. Conventional surgically implantable prosthetic valve typically include a leaflet assembly mounted within a relatively rigid support frame or ring. Components of the prosthetic valve are usually assembled with one or more biocompatible fabrics, and a fabric-covered sewing ring is provided around the valve for suturing to the tissue of the native leaflet.

Since surgeries are prone to an abundance of clinical complications, alternative less invasive techniques of delivering a prosthetic heart valve over a catheter and implanting it over the native malfunctioning valve have been developed over the years. Different types of prosthetic heart valves are known to date, including balloon expandable valve, self-expandable valves and mechanically-expandable valves. Different methods of delivery and implantation are also known, and may vary according to the site of implantation and the type of prosthetic valve. One exemplary technique includes utilization of a delivery assembly for delivering a prosthetic valve in a crimped state, from an incision which can be located at the patient's femoral or iliac artery, toward the native malfunctioning valve. Once the prosthetic valve is properly positioned at the desired site of implantation, it can be expanded against the surrounding anatomy, such as an annulus of a native valve, and the delivery assembly can be retrieved thereafter.

### SUMMARY

In a typical surgically implantable or catheter deliverable prosthetic valve, uniformly formed leaflets are sutured to the frame of the valve, either via an inner skirt that extends along the inner surface of the frame, or by suturing directly to struts of the frame. The leaflets are bent along the suture lines when transitioning to an open position during valve cycling, which can apply excessive stresses that can lead to leaflet tears at the points of suture penetration. Accordingly, improvements to devices and methods for securing leaflets or a leaflet structure to a frame of the valve are desirable.

The present disclosure is directed toward prosthetic heart valves that include non-uniform leaflet structures attached to a frame, the non-uniform leaflets structure including soft or malleable movable portions configured to freely transition between closed and open configurations, and stiffer portion(s) exhibiting increased stiffness or rigidity with respect to the movable portions.

According to one aspect of the disclosure, a prosthetic valve comprises a frame and a valvular structure coupled to the frame. The valvular structure comprises a plurality of non-uniform leaflets configured to regulate flow through the prosthetic valve. Each non-uniform leaflet comprises a movable body portion and at least one increased-stiffness portion.

In some aspects, the frame is movable between a radially compressed state and a radially expanded state.

In some aspects, the movable body portion is disposed between a free edge and an opposite cusp edge.

In some aspects, the at least one increased-stiffness portion is stiffer than the movable body portion.

In some aspects, each non-uniform leaflet is formed from a unitary continuous piece of material.

In some aspects, the at least one increased-stiffness portion comprises an inflow portion extending between the cusp edge and an inflow portion proximal end.

In some aspects, the frame comprises a plurality of intersecting struts, wherein the inflow portion is coupled to some of the struts of the frame.

In some aspects, the inflow portion defines an inflow portion width between the cusp edge and the inflow portion proximal end, wherein the struts to which the inflow portion is attached define a strut width between a strut inflow edge and a strut outflow edge, and wherein the inflow portion width is greater than the strut width.

In some aspects, each non-uniform leaflet further comprises a pair of oppositely-directed tabs between the cusp edge and the free edge.

In some aspects, the at least one increased-stiffness portion comprises tab stiff portions, each tab stiff portion extending from a tab outer edge of a corresponding tab to a tab portion inner boundary.

In some aspects, the at least one increased-stiffness portion comprises tab stiff portions, each tab stiff portion extending from a tab outer edge of a corresponding tab to a tab portion inner boundary.

In some aspects, each tab defines a tab length between the tab outer edge and an intersection of the tab with the cusp edge, wherein each tab stiff portion defines a stiff tab length between the tab outer edge and the tab portion inner boundary, and wherein the stiff tab length is greater than the tab length by an inner offsetting length.

In some aspects, the tabs of adjacent non-uniform leaflets are joined together to form commissures attached, directly or indirectly, to the frame.

In some aspects, the tab stiff portions are pre-shaped to assume a bent configuration in a free state prior to attachment commissure formation.

In some aspects, the tabs stiff portions extend radially inward from the frame along a length which is equal to or greater than the offsetting length.

In some aspects, the frame comprises commissure windows, wherein each commissure window comprises a commissure window opening extending between window sidewalls, and wherein each commissure is attached to a corresponding commissure window.

In some aspects, each of the tab stiff portion comprises a first section extending radially through a corresponding commissure window opening, and a second section folded sideways over an outer surface of the commissure window.

In some aspects, the frame comprises commissure support posts, and wherein each commissure is attached to a corresponding commissure support post.

In some aspects, each tab stiff portion comprises a first section extending radially from the tab portion inner boundary toward the commissure support post, a second section folded sideways over a support post inner surface, and a third section folded again to extend radially outward along at least a portion of a corresponding support post lateral side.

In some aspects, the prosthetic valve further comprises cell coupling members, wherein each cell coupling member extends across an opening of a cell formed by a plurality of interconnected angled struts of the frame, and wherein each commissure is attached to a corresponding cell coupling member.

In some aspects, the tab stiff portions are sutured to interconnected angled struts of the frame defining corresponding cells of the frame.

According to one aspect of the disclosure, a method of assembling a prosthetic valve comprises providing a plurality of non-uniform leaflets, each non-uniform leaflet comprising a movable body portion and at least one increased-stiffness portion, and attaching the at least one increased-stiffness portion of each non-uniform leaflet to a frame of the prosthetic valve.

In some aspects, each movable body portion is disposed between a free edge and an opposite cusp edge of the corresponding non-uniform leaflet.

In some aspects, the frame movable between a radially compressed state and a radially expanded state.

In some aspects, the at least one increased-stiffness portion is stiffer than the movable body portion.

In some aspects, each non-uniform leaflet is formed from a unitary continuous piece of material.

In some aspects, each non-uniform leaflet is formed from a unitary continuous piece of material.

In some aspects, the at least one increased-stiffness portion comprises an inflow portion extending between the cusp edge and an inflow portion proximal end.

In some aspects, attaching the at least one increased-stiffness portion of each non-uniform leaflet to the frame comprises attaching the inflow portion of each non-uniform leaflet to the frame.

In some aspects, each non-uniform leaflet further comprises a pair of oppositely-directed tabs between the cusp edge and the free edge.

In some aspects, the at least one increased-stiffness portion comprises tab stiff portions, each tab stiff portion extending from a tab outer edge of a corresponding tab to a tab portion inner boundary.

In some aspects, each tab defines a tab length between the tab outer edge and an intersection of the tab with the cusp edge, wherein each tab stiff portion defines a stiff tab length between the tab outer edge and the tab portion inner boundary, and wherein the stiff tab length is greater than the tab length by an inner offsetting length.

In some aspects, attaching the at least one increased-stiffness portion of each non-uniform leaflet to the frame comprises joining the tabs of adjacent non-uniform leaflets together to form commissures, and attaching the commissures to the frame.

In some aspects, the method further comprises

In some aspects, pre-shaping the tab stiff portions to assume a bent configuration in a free state prior to attaching the commissures to the frame.

According to some aspects of the disclosure, there is provided a prosthetic valve comprising a frame movable between a radially compressed state and a radially expanded state, and a valvular structure coupled to the frame and comprising a plurality of non-uniform leaflets configured to regulate flow through the prosthetic valve. Each non-uniform leaflet comprises a movable body portion disposed between a free edge and an opposite cusp edge, and at least one increased-stiffness portion. Each non-uniform leaflet is formed from a unitary continuous piece of material. The at least one increased-stiffness portion is stiffer than the movable body portion.

According to some aspects of the disclosure, there is provided a method of assembling a prosthetic valve, comprising a step of providing a plurality of non-uniform leaflets, each non-uniform leaflet comprising a movable body portion disposed between a free edge and an opposite cusp edge, and at least one increased-stiffness portion. The method further comprises a step of attaching the at least one increased-stiffness portion of each non-uniform leaflet to a frame movable between a radially compressed state and a radially expanded state. Each non-uniform leaflet is formed from a unitary continuous piece of material. The at least one increased-stiffness portion is stiffer than the movable body portion.

According to some aspects of the disclosure, there is provided a prosthetic valve comprising a frame movable between a radially compressed state and a radially expanded state, and a non-uniform valvular structure coupled to the frame and configured to regulate flow through the prosthetic valve. The frame comprises a plurality of vertical spikes. The non-uniform valvular structure comprises an inflow stiff portion disposed between a valvular distal edge and an inflow portion proximal boundary, and a movable body portion disposed between the inflow portion proximal boundary a free edge. The inflow stiff portion is stiffer than the movable body portion. The vertical spikes extend through the inflow stiff portion.

According to some aspects of the disclosure, there is provided a prosthetic valve comprising a frame and a non-uniform valvular structure coupled to the frame and configured to regulate flow through the prosthetic valve. The non-uniform valvular structure comprises a stiff portion and a plurality of movable regions. The stiff portions extends between a valvular distal edge and an undulating boundary. The stiff portion comprises a stiff inflow region and a plurality of stiff post regions continuously extending from the stiff inflow region. The movable regions are disposed between the undulating boundary and an outflow edge. The non-uniform valvular structure is formed of a unitary piece of material. The stiff portion is stiffer than the movable regions.

The aspects of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the invention will become more apparent from the following detailed description, which proceeds with reference to the accompanying figures.

### BRIEF DESCRIPTION OF THE FIGURES

Some examples of the invention are described herein with reference to the accompanying figures. The description, together with the figures, makes apparent to a person having ordinary skill in the art how some examples may be practiced. The figures are for the purpose of illustrative description and no attempt is made to show structural details of an example in more detail than is necessary for a fundamental understanding of the invention. For the sake of clarity, some objects depicted in the figures are not to scale.

In the Figures:

FIG. 1A is a perspective view of one example of a prosthetic valve.

FIG. 1B is a perspective view of the prosthetic valve of FIG. 1A, without the outer skirt.

FIG. 1C is a perspective view of a frame of the prosthetic valve of FIGS. 1A-1B.

FIG. 2 is a perspective view of an example of a leaflet structure that includes a thick sutured stitched along cusp edges of the leaflets.

FIG. 3 is an enlarged view of a portion of a prosthetic valve with the leaflet structure of FIG. 2 coupled to struts of its frame.

FIG. 4 is a side elevation view of a portion of a prosthetic valve, shown in a flattened configuration, demonstrating another manner by which a leaflet can be secured to the frame via strip.

FIG. 5 is a cross sectional view illustrating an exemplary coupling of a leaflet to the frame of FIG. 4 along the scallop line.

FIG. 6 is a cross sectional view illustrating an exemplary coupling of a valvular structure to a commissure window of the frame of FIGS. 1A-1B.

FIG. 7 is a side elevation view of an example of a non-uniform leaflet, shown in a flattened configuration.

FIG. 8 is a cross sectional view illustrating an exemplary coupling of a non-uniform leaflet to the frame of a prosthetic valve along the scalloped line.

FIG. 9 is a detail view of a portion of a prosthetic valve with non-uniform leaflets forming an exemplary commissure coupled to a commissure window of the frame of FIG. 1C.

FIG. 10 is a cross sectional view illustrating one exemplary configuration of coupling a commissure of non-uniform leaflets to a commissure window.

FIG. 11 is a cross sectional view illustrating another exemplary configuration of coupling a commissure of non-uniform leaflets to a commissure window.

FIG. 12 is a detail view of a portion of a prosthetic valve with non-uniform leaflets forming an exemplary commissure coupled to an open commissure window.

FIG. 13 is a cross sectional view illustrating an exemplary configuration of coupling a commissure of non-uniform leaflets to a commissure window equipped with holes extending through the window sidewalls.

FIG. 14 is a simplified perspective view of another exemplary configuration of coupling a commissure of non-uniform leaflets to a commissure window equipped with holes extending through the window sidewalls.

FIG. 15 is a simplified perspective view of an exemplary configuration of coupling a commissure comprised of uniform leaflets to a commissure support post.

FIG. 16 is a cross sectional view illustrating an exemplary configuration of coupling a commissure of non-uniform leaflets to a commissure support post.

FIG. 17A is a detail, perspective view of another exemplary configuration of coupling a commissure to a cell coupling member attached to a cell of the frame.

FIG. 17B is a detail, perspective view of another exemplary configuration of coupling a commissure directly to angled struts of a frame cell.

FIG. 18 is a side elevation view of an example of a non-uniform valvular structure, shown in an unrolled or flattened configuration.

FIG. 19 is a side elevation view of an example of a single non-uniform leaflet that can be used to form a non-uniform valvular structure, shown in a flattened configuration.

FIG. 20 is a side elevation view of an exemplary frame with vertical spikes, shown in a flattened configuration.

FIG. 21 is a perspective view from the inside of a portion of a prosthetic valve including a non-uniform valvular structure attached to a frame of the type shown in FIG. 20.

FIG. 22 is a cross-sectional view taken along line 22-22 of FIG. 21.

FIG. 23 is a side elevation view of another exemplary frame with two sets of vertical spikes, shown in a flattened configuration.

FIG. 24 shows an exemplary delivery apparatus carrying a balloon expandable prosthetic valve.

FIG. 25 is a perspective view of an exemplary conventional surgically implantable prosthetic valve with portions cutaway to reveal internal structural components thereof.

FIG. 26 is a side elevation view of an exemplary non-uniform valvular structure, shown in an unrolled or flattened configuration, prior to pre-shaping thereof.

FIG. 27 shows a disassembled view of a surgically-implantable prosthetic valve that includes a pre-shaped non-uniform valvular structure.

FIG. 28 shows is a perspective view of the prosthetic valve of FIG. 27 in an assembled configuration.

### DETAILED DESCRIPTION

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, can be used in combination with any other feature described herein.

As used in this application and in the claims, the singular forms “a,” “an,” and “the” include the plural forms unless the context clearly dictates otherwise. Additionally, the terms “have” or “includes” means “comprises.” Further, the term “coupled” generally means physically, mechanically, chemically, magnetically, and/or electrically coupled or linked and does not exclude the presence of intermediate elements between the coupled or associated items absent specific contrary language. As used herein, “and/or” means “and” or “or,” as well as “and” and “or”.

Directions and other relative references may be used to facilitate discussion of the drawings and principles herein, but are not intended to be limiting. For example, certain terms may be used such as “inner,” “outer,” “upper,” “lower,” “inside,” “outside,”, “top,” “bottom,” “interior,” “exterior,” “left,” right,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

Throughout the figures of the drawings, different superscripts for the same reference numerals are used to denote different examples of the same elements. Examples of the disclosed devices and systems may include any combination of different examples of the same elements. Specifically, any reference to an element without a superscript may refer to any alternative example of the same element denoted with a superscript. In order to avoid undue clutter from having too many reference numbers and lead lines on a particular drawing, some components will be introduced via one or more drawings and not explicitly identified in every subsequent drawing that contains that component.

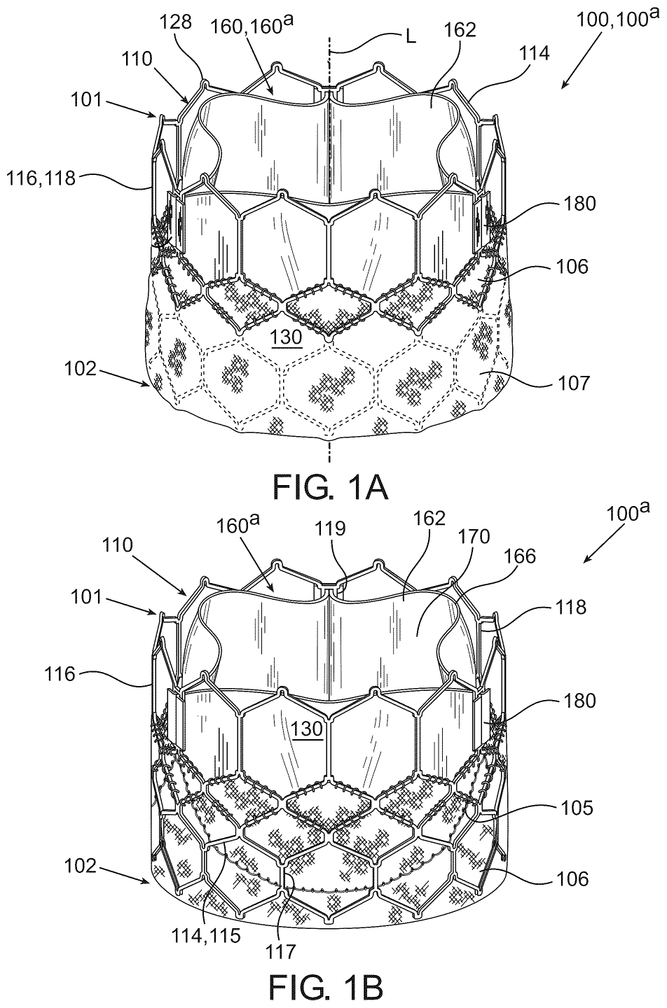

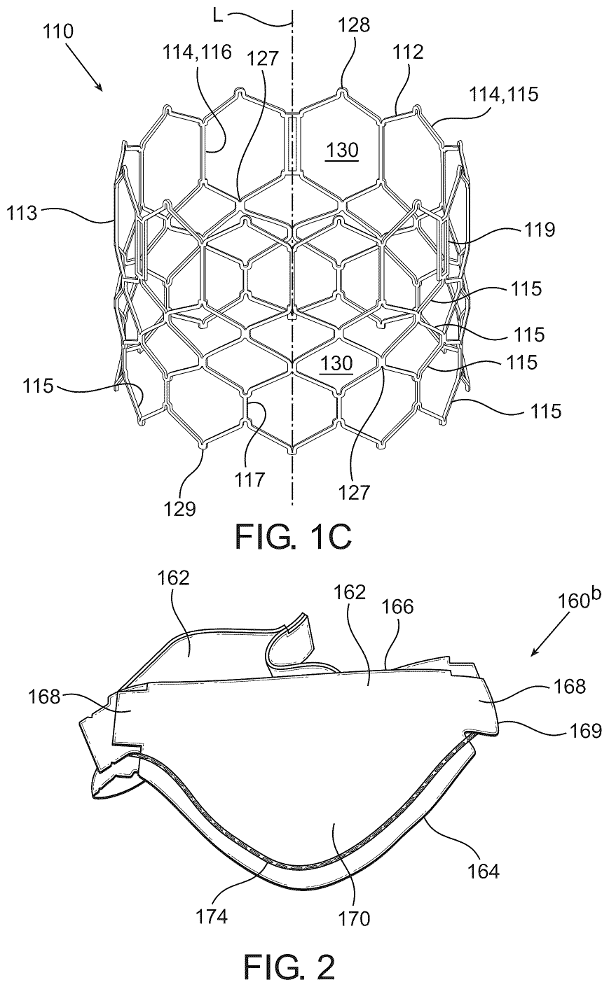

FIG. 1A and 1B show perspective views of one example of a prosthetic valve 100, with and without an outer skirt 107 surrounding the frame 110, respectively. FIG. 1C shows the frame 110 without any other soft components attached thereto. The term “prosthetic valve”, as used herein, refers to any type of a prosthetic valve deliverable to a patient's target site over a catheter, which is radially expandable and compressible between a radially compressed, or crimped, state, and a radially expanded state. Thus, the prosthetic valve can be crimped on or retained by an implant delivery apparatus (not shown) in the radially compressed state during delivery, and then expanded to the radially expanded state once the prosthetic valve reaches the implantation site. The expanded state may include a range of diameters to which the valve may expand, between the compressed state and a maximal diameter reached at a fully expanded state. Thus, a plurality of partially expanded states may relate to any expansion diameter between radially compressed or crimped state, and maximally expanded state. A prosthetic valve of the current disclosure (e.g., prosthetic valve 100, 300) may include any prosthetic valve configured to be mounted within the native aortic valve, the native mitral valve, the native pulmonary valve, and the native tricuspid valve.

It is understood that the prosthetic valves disclosed herein may be used with a variety of implant delivery apparatuses. Balloon expandable valves generally involve a procedure of inflating a balloon within a prosthetic valve, thereby expanding the prosthetic valve within the desired implantation site. Once the valve is sufficiently expanded, the balloon is deflated and retrieved along with a delivery apparatus (not shown). Self-expandable valves include a frame that is shape-set to automatically expand as soon an outer retaining shaft or capsule (not shown) is withdrawn proximally relative to the prosthetic valve. Mechanically expandable valves are a category of prosthetic valves that rely on a mechanical actuation mechanism for expansion. The mechanical actuation mechanism usually includes a plurality of expansion and locking assemblies (such as the prosthetic valves described in U.S. Pat. No. 10,603,165, International Application No. PCT/US2021/052745 and U.S. Provisional Application Nos. 63/085,947 and 63/209,904, each of which is incorporated herein by reference in its entirety), releasably coupled to respective actuation assemblies of a delivery apparatus, controlled via a handle (not shown) for actuating the expansion and locking assemblies to expand the prosthetic valve to a desired diameter. The expansion and locking assemblies may optionally lock the valve's diameter to prevent undesired recompression thereof, and disconnection of the actuation assemblies from the expansion and locking assemblies, to enable retrieval of the delivery apparatus once the prosthetic valve is properly positioned at the desired site of implantation.

The term “plurality”, as used herein, means more than one.

FIGS. 1A-1C show an example of a prosthetic valve 100, which can be a balloon expandable valve, illustrated in an expanded state. The prosthetic valve 100 can comprise an outflow end 101 and an inflow end 102. In some instances, the outflow end 101 is the proximal end of the prosthetic valve 100, and the inflow end 102 is the distal end of the prosthetic valve 100. Alternatively, depending for example on the delivery approach of the valve, the outflow end can be the distal end of the prosthetic valve, and the inflow end can be the distal end of the proximal valve.

The term “proximal”, as used herein, generally refers to a position, direction, or portion of a device or a component of a device, which is closer to the user (for example, closer to an operator of a delivery apparatus utilized during an implantation procedure) and farther away from the implantation site.

The term “distal”, as used herein, generally refers to a position, direction, or portion of a device or a component of a device, which is farther away from the user and closer to the implantation site.

The term “outflow”, as used herein, refers to a region of the prosthetic valve through which the blood flows through and out of the prosthetic valve 100.

The term “inflow”, as used herein, refers to a region of the prosthetic valve through which the blood flows into the prosthetic valve 100.

In the context of the present application, the terms “lower” and “upper” are used interchangeably with the terms “inflow” and “outflow”, respectively. Thus, for example, the lower end of the prosthetic valve is its inflow end and the upper end of the prosthetic valve is its outflow end.

In the context of the present application, the terms “lower” and “upper” are used interchangeably with the terms “distal to” and “proximal to”, respectively. Thus, for example, a lowermost component can refer to a distal-most component, and an uppermost component can similarly refer to a proximal-most component.

The terms “longitudinal” and “axial”, as used herein, refer to an axis extending in the proximal and distal directions, unless otherwise expressly defined.

The prosthetic valve 100 comprises an annular frame 110 movable between a radially compressed configuration and a radially expanded configuration, and a valvular structure 160 mounted within the frame 110. The frame 110 can be made of various suitable materials, including plastically-deformable materials such as, but not limited to, stainless steel, a nickel based alloy (e.g., a cobalt-chromium or a nickel-cobalt-chromium alloy such as MP35N alloy), polymers, or combinations thereof. When constructed of a plastically-deformable materials, the frame 110 can be crimped to a radially compressed state on a balloon catheter, and then expanded inside a patient by an inflatable balloon or equivalent expansion mechanism. Alternatively or additionally, the frame 110 can be made of shape-memory materials such as, but not limited to, nickel titanium alloy (e.g., Nitinol). When constructed of a shape-memory material, the frame 110 can be crimped to a radially compressed state and restrained in the compressed state by insertion into a shaft or equivalent mechanism of a delivery apparatus.

In the example illustrated in FIGS. 1A-1C, the frame 110 is an annular, stent-like structure comprising a plurality of intersecting struts 114. In this application, the term “strut” encompasses axial struts, angled struts, laterally extendable struts, commissure windows, commissure support struts, support posts, and any similar structures described by U.S. Pat. Nos. 7,993,394 and 9,393,110, which are incorporated herein by reference. A strut 114 may be any elongated member or portion of the frame 110. The frame 110 can include a plurality of strut rungs that can collectively define one or more rows of cells 130. The frame 110 can have a cylindrical or substantially cylindrical shape having a constant diameter from the inflow end 102 to the outflow end 101 as shown, or the frame can vary in diameter along the height of the frame, as disclosed in U.S. Pat. No. 9,155,619, which is incorporated herein by reference.

The end portions of the struts 114 are forming apices 128 at the outflow end 101 and apices 129 at the inflow end 102. The struts 114 can intersect at additional junctions 127 formed between the outflow apices 128 and the inflow apices 129. The junctions 127 can be equally or unequally spaced apart from each other, and/or from the apices 128, 129, between the outflow end 101 and the inflow end 102.

The struts 114 can include a plurality of angled struts 115 and vertical or axial struts 116. FIGS. 1A-1C show an exemplary prosthetic valve 100 that can be representative of, but is not limited to, a balloon expandable prosthetic valve. The frame 110 of the prosthetic valve 100 illustrated in FIG. 1C comprises rungs of angled struts 115 and axial struts 116 disposed between some of the rungs of the angled struts. In such implementations of the frame, the struts can be pivotable or bendable relative to each other, so as to permit frame expansion or compression. For example, the frame 110 can be formed from a single piece of material, such as a metal tube, via various processes such as, but not limited to, laser cutting, electroforming, and/or physical vapor deposition, while retaining the ability to collapse/expand radially in the absence of hinges and like.

A conventional valvular structure 160, shown also for example in FIG. 2, can include a plurality of leaflets 162 (e.g., three leaflets), positioned at least partially within the frame 110, and configured to regulate flow of blood through the prosthetic valve 100 from the inflow end 102 to the outflow end 101. While three leaflets 162 arranged to collapse in a tricuspid arrangement, are shown in the example illustrated in FIGS. 1A-1B and 2, it will be clear that a prosthetic valve 100 can include any other number of leaflets 162. Adjacent leaflets 162 can be arranged together to form commissures 180 that are coupled (directly or indirectly) to respective portions of the frame 110, thereby securing at least a portion of the valvular structure 160 to the frame 110. The leaflets 162 can be made from, in whole or part, biological material (e.g., pericardium), bio-compatible synthetic materials, or other such materials. Further details regarding transcatheter prosthetic heart valves, including the manner in which the valvular structures 160 can be coupled to the frame 110 of the prosthetic valve 100, can be found, for example, in U.S. Pat. Nos. 6,730,118, 7,393,360, 7,510,575, 7,993,394, 8,652,202, and 11,135,056, all of which are incorporated herein by reference in their entireties.

As shown for example in FIG. 2, three separate leaflets 162 can collectively define the valvular structure 160 in some cases. Each conventional separate leaflet 162 can have a rounded cusp edge 164 opposite a free edge 166, and a pair of generally oppositely-directed tabs 168 separating the cusp edge 164 and the free edge 166. The cusp edge 164 in such cases forms a single scallop. Each separate leaflet 162 further comprises an inner surface (not annotated), defined as the surface facing the valve central longitudinal axis L, and an outer surface (not annotated), opposite thereto so as to face the frame 110.

When such leaflets 162 are coupled to the frame and to each other, the lower edge of the resulting valvular structure 160 desirably has an undulating, curved scalloped shape. By forming the leaflets with this scalloped geometry, stresses on the leaflets 162 are reduced which, in turn, improves durability of the prosthetic valve. Moreover, by virtue of the scalloped shape, folds and ripples at the belly of each leaflet, which can cause early calcification in those areas, can be eliminated or at least minimized. The scalloped geometry also reduces the amount of tissue material used to form the valvular structure, thereby allowing a smaller, more even crimped profile at the inflow end of the valve.

The leaflets 162 define a non-planar coaptation plane (not annotated) when their free edges 166 co-apt with each other to seal blood flow through the prosthetic valve 100. Leaflets 162 can be secured to one another at their tabs 168 to form commissures 180 of the valvular structure 160, which can be secured, directly or indirectly, to structural elements connected to the frame 110 or integrally formed as portions thereof, such as commissure posts, commissure windows, and the like. When secured to two other leaflets 162 to form valvular structure 160, the cusp edges 164 of the leaflets 162 collectively form the scalloped line 105 of the valvular structure 160. Each leaflet 162 comprises a leaflet body 170, defined between the line of attachment of the leaflet to the frame, for example along scalloped line 105, and the free edge 166. The leaflet body 170 defines the movable portion of the leaflet 162, free to move toward the frame 110 in an open state of the valvular structure 160, and toward central longitudinal axis L to co-apt with other leaflets 162 in a closed state of the valvular structure 160.

In some examples, the prosthetic valve 100 can further comprise at least one skirt or sealing member. FIGS. 1A-1B show an example of a prosthetic valve 100^{a} that includes an inner skirt 106, which can be secured to the inner surface 112 of the frame 110. Such an inner skirt 106 can be configured to function, for example, as a sealing member to prevent or decrease perivalvular leakage. An inner skirt 106 can further function as an anchoring region for valvular structure 160^{a} to the frame 110, and/or function to protect the leaflets 162 against damage which may be caused by contact with the frame 110, for example during valve crimping or during working cycles of the prosthetic valve 100. FIG. 1B shows an inner skirt 106 disposed around and attached to the inner surface 112 of frame 110, wherein the valvular structure 160a is sutured to the inner skirt 106 along scalloped line 105. Additionally, or alternatively, the prosthetic valve 100 can comprise an outer skirt 107 mounted on the outer surface 113 of frame 110, configure to function, for example, as a sealing member retained between the frame 110 and the surrounding tissue of the native annulus against which the prosthetic valve is mounted, thereby reducing risk of paravalvular leakage (PVL) past the prosthetic valve 100.

Any of the inner skirt 106 and/or outer skirt 107 can be made of various suitable biocompatible materials, such as, but not limited to, various synthetic materials (e.g., PET) or natural tissue (e.g. pericardial tissue). In some cases, the inner skirt 106 can be formed of a single sheet of material that extends continuously around the inner surface 112 of frame 110. In some examples, the outer skirt 107 can be formed of a single sheet of material that extends continuously around the outer surface 113 of frame 110.

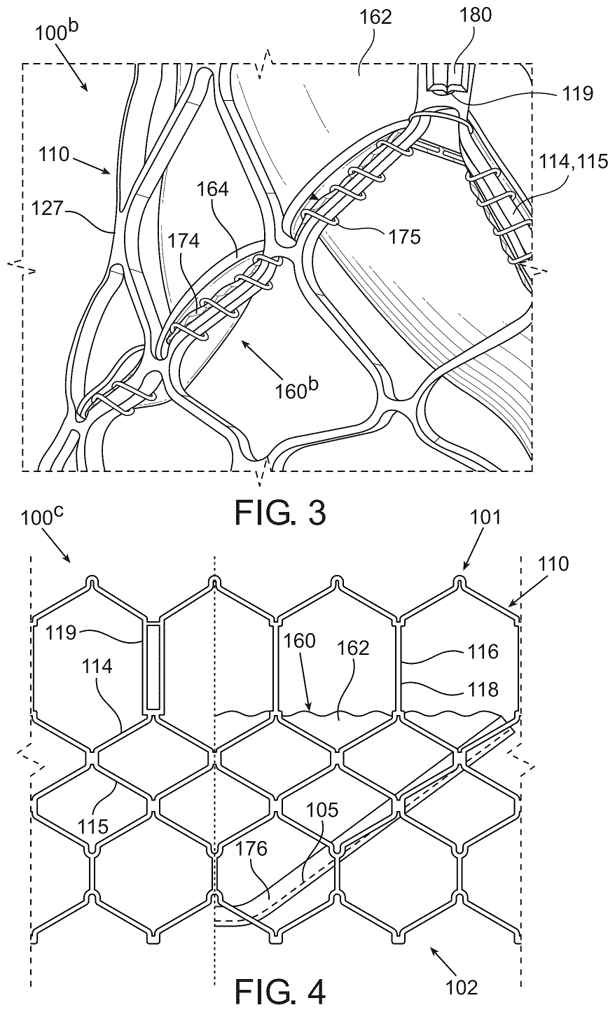

FIG. 3 shows a zoomed in view of another attachment configuration of a valvular structure 160^{b}, shown in FIG. 2, to a frame 110 of a prosthetic valve 100^{b}, in which the leaflet end portions adjacent cusp edges 164 are sutured to struts 114 that can generally follow the contour of the cusp edges 164, in a manner that can allow for the elimination of an inner skirt in the assembled prosthetic valve. Reducing the number of soft components of the prosthetic valve (e.g., by eliminating the inner skirt and suturing the leaflets directly to the frame) can simplify a process for assembling the prosthetic valve. For example, assembling prosthetic valves including an inner skirt can result in a prolonged assembly time which involves suturing each of the leaflets to the inner skirt and then suturing the inner skirt to the frame of the prosthetic valve.

FIG. 2 shows separate leaflets 162 secured together into an exemplary valvular structure 160^{b} with a thick suture line 174 disposed adjacent cusp edge 164 and tracking the contour of cusp edge 164 of each of the leaflets 162. In such examples, a connecting suture line 174 can enable securing of the valvular structure 160^{b} directly to a frame 110, without having to extend sutures through the leaflet during assembly to the frame. As shown in FIG. 3, direct attachment of the valvular structure 160 of FIG. 2 to the frame 110 can be achieved by looping a connecting suture 175 around the struts 114 and through or around portions of the thick suture 174.

FIG. 1C shows the frame 110 of the prosthetic valve 100 with the other components, such as leaflets and skirts, removed. FIG. 1C shows the frame 110 in an annular configuration, corresponding to its functional configuration, while FIG. 4 shows a portion of the frame 110 in a flat configuration for purposes of illustration. The frame 110 can comprise, in some examples, a plurality of rows or rungs of angled struts, as well as axial struts that can extend between some rungs of angled struts. The struts 114 collectively define a plurality of cells 130 of the frame 110.

In the example illustrated in FIG. 1C, the frame 110 comprises five rungs of circumferentially extending angled struts 115. A plurality of substantially straight distal axial struts 117 can extend from junctions 127 of angled struts 115 at the inflow end 102 of the valve. Similarly, a plurality of substantially straight proximal axial struts, which can be either proximal non-windowed axial struts 118 or axial struts that include commissure windows 119, can extend from junctions 127 of angled struts 115 at the outflow end 101 of the valve.

The axial length of the proximal axial struts can be different than that of the distal axial struts. For example, in the illustrated configuration the proximal non-windowed axial struts 118, as well as axial struts 116 that include commissure windows 119, can be longer than the distal axial struts 117. In some examples, at least some (e.g., three) of the proximal axial struts 116 can define axially extending window frame portions, also termed commissure windows 119, configured to mount respective commissures 180 of the valvular structure 160. A commissure window 119 can include a commissure window opening 122 extending radially through the thickness of the commissure window 119, between window sidewalls 120, as shown for example in FIG. 6. The commissure window opening 122 can be configured to accept tabs 168 therein to couple the valvular structure 160 to the frame 110.

In some examples, the leaflets 162 of a valvular structure 160 can be coupled to the frame 110 via a strip 176. FIG. 4 shows a flattened portion of an exemplary prosthetic valve 100^{c}, in which a leaflet 162 is secured along its cusp edge 164 to the frame 110 via a strip 176. The strip 176 can be provided as an elongate, generally rectangular component, comprising any suitable synthetic material (e.g., PET) or natural tissue.

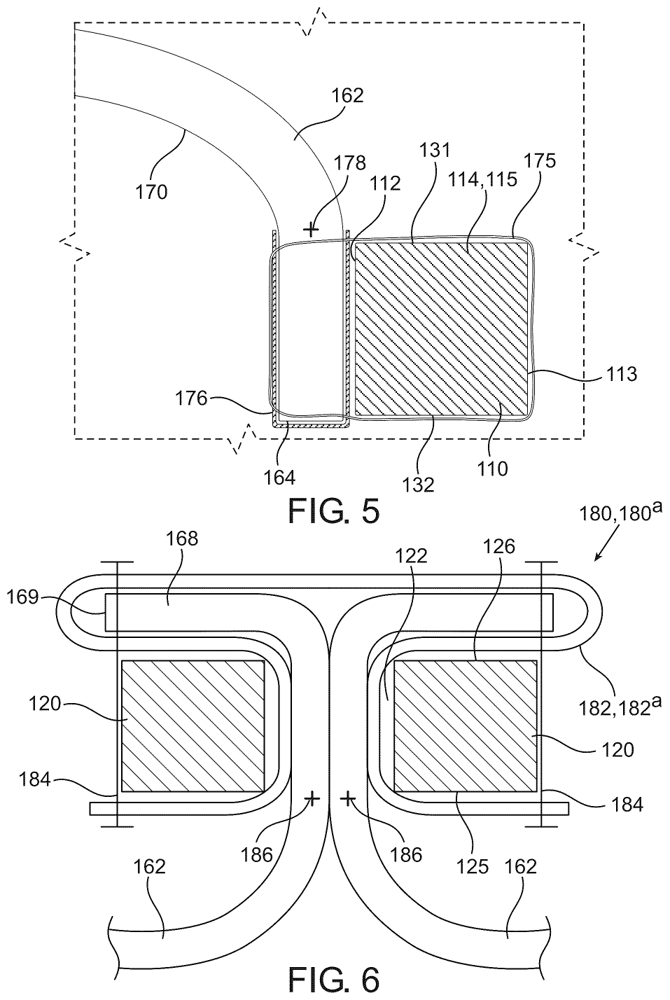

FIG. 5 shows an exemplary cross-sectional view of a portion of the leaflet 162 coupled to a frame 110 along scalloped line 105 (see FIG. 4), wherein a strip 176 can be folded over the cusp edge 164 to extend and cover both sides of the leaflet 162 along a distance that can be, in some examples, slightly greater than the height of a strut 115. A connecting suture 175 is looped around the strut 114 of the frame 110, which can be an angled strut 115, extending over the outer surface 113 of the frame 110, along the strut outflow edge 131, through an outer layer of the strip 176 (disposed between the leaflet 162 and the frame 110) and the thickness of the leaflet 162, further extending therefrom along an inner layer of the strip 176 (facing the central longitudinal axis L), back through the inner layer of the strip 176 and the leaflet 162, outward along the strut inflow edge 132 toward the outer surface 113 of the frame 110.

During valve cycling, the leaflets can articulate about inflow bending lines 178, between an open state during systole and coaptation state during diastole. The bending lines 178 of the leaflets 162 can follow the scallop line 105, and form adjacent to the upper or proximal penetration points of the connecting suture 175 into leaflets 162, which can be also referred to as bending lines 178 formed adjacent the inflow edge 132 of the struts 114 they are attached to. The stresses generated on the leaflet tissue by such bending of the leaflets proximate the suture penetration points into the leaflet can lead to fatigue failure of the leaflet.

FIG. 6 illustrates one example of commissure 180^{a} coupled to a commissure window 119 of a frame 110. In the illustrated example, tabs 168 of adjacent leaflet 162 can extend through the commissure window opening 122 and can be folded along the radially outer surface 126 of the commissure window. If the commissure window 119 is integrally formed with an axial strut 116 of the frame 110, the commissure window outer surface 126 is the outer surface 113 of the frame 110, and the commissure window inner surface 125 is the inner surface 112 of the frame 110. However, in some examples, a commissure window 119 can be formed within a separate strut or post member which is attachable to the frame 110, and can be disposed radially inward to, or radially outward to, the frame 110. In such cases, the commissure window 119 will exhibit a commissure window outer surface 126 and a commissure window inner surface 125 which are not necessarily aligned with the frame's outer surface 113 and inner surface 112, respectively.

As further shown in FIG. 6, a commissure coupling member 182 (e.g., a flexible connector 182^{a} comprising a fabric) can extend along the commissure window inner surface 125, through the commissure window opening 122, around the outer edges 169 of each tab 168, and across the radially outer surface of the tabs 168, such that the commissure coupling member 182^{a} forms a plurality of layers (e.g., three layers in the illustrated example). The various components can be coupled together using one or more sutures 184. In the illustrated example, each suture 184 extends through two outer layers of the commissure coupling member 182^{a}, through the tab 168, and through an inner layer of the commissure coupling member 182^{a}. Further details regarding commissure tab assemblies and additional commissure configurations useable with frame 110 can be found, at least, in U.S. Pat. Nos. 9,393,110, 11,026,785 and 11,135,056, and U.S. Provisional Application No. 63/049,812, which are all incorporated by reference herein in their entireties.

During valve cycling, the leaflets can articulate about commissure bending axes 186, between an open state during systole and coaptation state during diastole. The bending axes 186 of the leaflets 162 can at or near the level of the inner surface 112 of the frame 110, which can cause significant portion of the leaflets 162 to contact and/or hit against the inner surface 112 of the frame 110 during their open state. Repetitive contact of the leaflets 162 with the frame 110 as the leaflets open can damage, weaken, and/or cause wear to the leaflets over time.

Valvular structures 160 of conventional prosthetic valves, such as prosthetic valve 100, include leaflets 162 exhibiting uniform material stiffness, also referred to herein as uniform valvular structures 160 that include uniform leaflets 162. The terms “uniform” or “uniform material stiffness”, as used herein, are interchangeable and refer to a material having uniform or homogenous material properties, and specifically, uniform stiffness, along its entire surface and/or volume. It is to be understood that a uniform material stiffness of a leaflet refers to the material properties of the base material (which can be made of tissue, such as pericardial tissue), without any other components that can be attached thereto, such as sutures, fabrics, strips and the like. In contrast, the term “non-uniform” or “non-uniform material stiffness”, as used herein, are interchangeable and refer to a material having different material properties, and specifically, different stiffness, in different regions or portions thereof. The stiffness of any portion of a valvular structure and/or a leaflet can be measured by following the general procedures set forth in ASTM D790.

As described above, leaflets 162 can be secured along their inflow ends to the frame via various intermediate components, such as by being sutured to an inner skirt 106, as illustrated and described for a prosthetic valve 100^{a}, by being sutured directly to struts 114 of the frame 110 along a thick suture line 174, as illustrated and described for a prosthetic valve 100^{b}, or by being coupled to the frame via a strip 176 that can be folded over the cusp edges 164, as illustrated and described for a prosthetic valve 100^{c}. All of these exemplary methods of attachment include sutures that penetrate into the tissue material of the leaflet, wherein the resulting inflow bending lines 178 may be formed at or in close proximity to the points of suture penetration. As also described above, leaflets 162 can be joined together at their tabs 168 and coupled to the frame, for example extending through a commissure window 119 as illustrated and described for commissure 180^{a}, wherein the resulting commissure bending axes 186 may result in repetitive contact of the leaflets with the frame 110 in a manner that can subject the leaflets to wear damage over time. All of these factors may adversely impact the functionality and structural integrity of the valvular structure 160 in the long term. Described below are non-homogenous valvular structures, optionally including non-homogenous leaflets, provided with increased-stiffness portions that can offset the inflow bending lines 178 and/or the commissure bending axes 186, so as to improve long-term durability of the valvular structure.

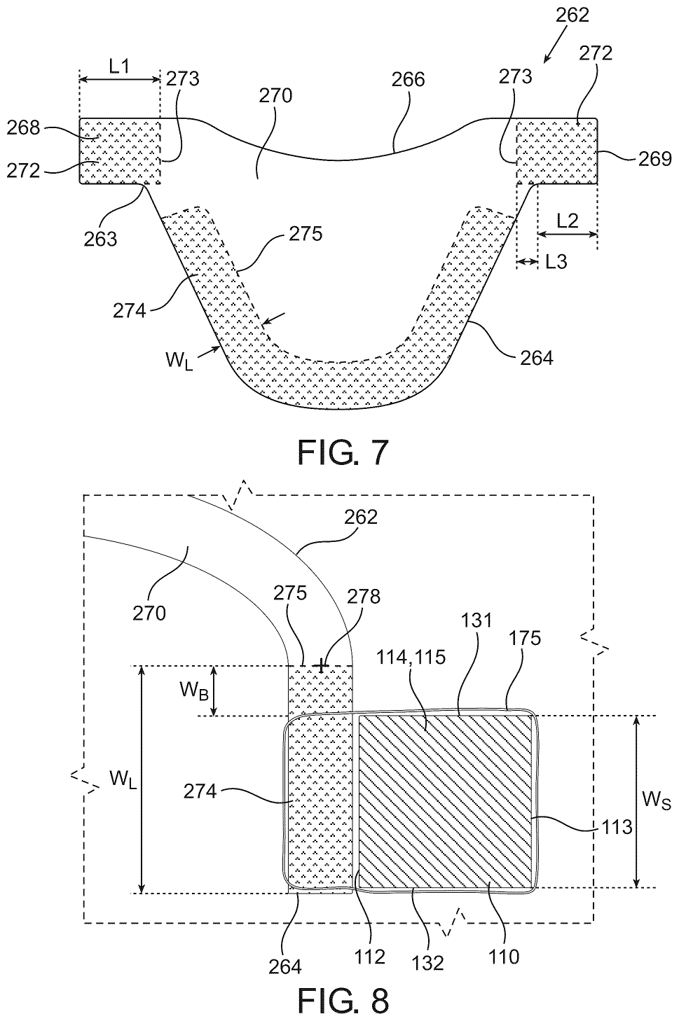

FIG. 7 shows an example of a non-uniform leaflet 262, which can be generally similar to leaflet 162, including similar tabs 268 disposed between a cusp edge 264 and a free edge 266, with the main difference being that the non-uniform leaflet 262 has a movable body portion 270 and at least one additional increased-stiffness region or portion provided with a stiffness that is greater than the stiffness of the movable body portion 270. In some examples, the non-uniform leaflet 262 includes an inflow portion 274 extending from the cusp edge 264 and terminating at an inflow portion proximal end 275, wherein the inflow portion 274 is stiffer than the movable body portion 270. Thus, in such examples, the at least one increased-stiffness portion includes the inflow portion 274. The inflow portion proximal end 275 can be the border between the movable body portion 270 and the inflow portion 274, defining an inflow portion width WL between the cusp edge 264 and the inflow portion proximal end 275.

The inflow portion 274 can extend all the way up to the tabs 268, or it can terminate below the level of the tabs, as illustrated in FIG. 7. In some examples, the inflow portion proximal end 275 is parallel to the cusp edge 264, such that the inflow portion width WL is uniform along the inflow portion 274. In other examples, the inflow portion width WL can vary, for example from a wider width at the lower tip of the leaflet to a narrower width at the upper ends of the inflow portion 274, closer to tabs 268.

In some examples, the non-uniform leaflet 262 includes tab stiff portions 272 extending along the tabs 268 from the tab outer edges 269 to tab portion inner boundaries 273, wherein the tab stiff portions 272 are stiffer than the movable body portion 270. Thus, in such examples, the at least one increased-stiffness portion includes the tab stiff portions 272. Each tab portion inner boundary 273 can be a border between the movable body portion 270 and the corresponding tab stiff portion 272, defining a stiff tab length L1 between the tab outer edge 269 and the tab portion inner boundary 273. The length L2 of tab 268 can be defined as the length between the outer edge 269 and the transition zone 263 between the tabs lower edge and the cusp edge 264. In some examples, the stiff tab length L1 is equal to the tab length L2. In other examples, as illustrated in FIG. 7, the stiff tab length L1 is greater than the tab length L2, such that the tab stiff portion 272 extends further beyond the length L2 to an inner offsetting length L3, that is to say that L1=L2+L3 (wherein L3>0).

In some examples, the tab portion inner boundary 273 is parallel to the tab outer edge 269, such that the stiff tab length L1 is uniform along the height of tab 268. In other examples, the tab portion inner boundary 273 is not necessarily parallel to the tab outer edge 269, in which case, stiff tab length L1 is defined as the maximal length between the tab outer edge 269 and the point along tab portion inner boundary 273 which is farthest therefrom.

A non-uniform leaflet 262 is formed from a unitary continuous piece of material, wherein different portions thereof can be treated to have different material properties. For example, at least one increased-stiffness portion of a non-uniform leaflet 262, such as an inflow portion 274 and/or tab stiff portions 272, is stiffer than movable body portion 270. A non-uniform leaflet 262 can be formed from natural tissue, such as bovine pericardium, undergoing biological treatment procedures that can include subjecting the tissue to cross-linking agents, which can influence its final material properties. In such an example, each of the movable body portion 270 and the inflow portion 274 and/or tab stiff portions 272 can be subjected to a different biological procedure, including being subjected to different cross-linking agents, subjected to such agents for different time durations, or other procedural variations, adapted to result in an inflow portion 274 and/or tab stiff portions 272 that can be stiffer than the corresponding movable body portion 270. Thus, the movable body portion 270, which has material properties comparable to those of the leaflet body 170 of conventional leaflets 162, is the portion of the leaflet 262 which is not directly attached to the frame 110, but is rather configured to freely move toward the frame 110 in an open state of the valvular structure 260, and toward central longitudinal axis L in a closed state of the valvular structure 260.

As mentioned, a non-uniform leaflet 262 can optionally exhibit a different degree of cross-linking in different portions thereof, such that the inflow portion 274 and/or tab stiff portions 272 can be more highly cross-linked than the movable body portion 270. This can be achieved, in some examples, by cross-linking the inflow portion 274 and/or tab stiff portions 272 with a first cross-linking agent or solution and cross-linking the movable body portion 270 with a second cross-linking agent. Alternatively or additionally, the inflow portion 274 and/or tab stiff portions 272 can be cross-linked during a longer period of time relative to the movable body portion 270. Cross-linking agents can include, but are not limited to, divinyl sulfone (DVS), polyethylene glycol divinyl sulfone (VS-PEG-VS), hydroxyethyl methacrylate divinyl sulfone (HEMA-DIS-HEMA), formaldehyde, glutaraldehyde, aldehydes, isocyanates, alkyl and aryl halides, imidoesters, N-substituted maleimides, acylating compounds, carbodiimide, hexamethylene diisocyanate, 1-Ethyl-3-[3-dimethylaminopropyl]carbodiimide hydrochloride (EDC or EDAC), hydroxychloride, N-hydroxysuccinimide, and combinations thereof.

The movable body portion 270 can be designed to have material properties substantially similar to those of conventional leaflets 162 described above with respect to FIGS. 1A-3, allowing this portion to move between the closed and open states in a similar manner. The inflow portion 274 can be stiffer compared to the movable body portion 270, facilitating easier penetration of needles utilized to suture these portions to the frame during valve assembly, and improving engagement retention with the sutures. The inflow portion 274 can be semi-rigid, meaning that it is stiffer or more rigid than the movable body portion 270, yet flexible enough to transition between the crimped and expanded configurations of the prosthetic valve without experiencing material failure and without resisting such transitions of the valve.

In some examples, the ultimate tensile stress of the increased-stiffness portion(s), such as the inflow portion 274 and/or tab stiff portions 272, is at least 1.5 times greater than the ultimate tensile stress of the movable body portion 270. In some examples, the ultimate tensile stress of the inflow portion 274 and/or tab stiff portions 272, is at least 2 times greater than the ultimate tensile stress of the movable body portion 270. For example, for a movable body portion 270 having an ultimate tensile stress of about 1 MPa (Megapascal), the inflow portion 274 and/or tab stiff portions 272 can have an ultimate tensile stress which is greater than 1.5 MPa, and in some examples, greater than 2 MPa.

In some examples, the increased-stiffness portion(s), such as the inflow portion 274 and/or tab stiff portions 272, obtain a load at failure which is at least 2 times greater than the load at failure obtained by the movable body portion 270. In some examples, the inflow portion 274 and/or tab stiff portions 272 obtain a load at failure which is at least 3 times greater than the load at failure obtained by the movable body portion 270. For example, for a movable body portion 270 that obtains a load at failure of about 3 N (Newton), the inflow portion 274 and/or tab stiff portions 272 can obtain a load at failure which is greater than 6 N, and in some examples, greater than 9 N.

While in some examples, a non-uniform leaflet 262 can include both stiffer inflow portion 274 and tab stiff portions 272, as illustrated in FIG. 7, it is to be understood that this is not meant to be limiting. For instance, in some examples, a non-uniform leaflet 262 can include only stiffer inflow portion 274 while the tabs 268 have the same stiffness as that of the movable body portion 270. In some examples, a non-uniform leaflet 262 can include only tab stiff portions 272 without a stiffer inflow portion.

When a non-uniform leaflet 262 includes both a stiffer inflow portion 274 and tab stiff portions 272, their stiffness can be similar or different. For example, while both the inflow portion 274 and tab stiff portions 272 will be stiffer than the movable body portion 270, the inflow portion 274 can be stiffer than tab stiff portions 272 in some examples, or the tab stiff portions 272 can be stiffer than the inflow portion 274 in other examples.

For any of a stiffer inflow portion 274 and/or tab stiff portions 272, their stiffness can be homogenous or non-homogenous. For example, the stiffness of the inflow portion 274 can be homogenous between the cusp edge 264 and the inflow portion proximal end 275, or it can vary from higher stiffness at the cusp edge to lower stiffness at the inflow portion proximal end 275. Similarly, the stiffness of each tab stiff portion 272 can be homogenous between the tab outer edge 269 and the tab portion inner boundary 273, or it can vary from higher stiffness at the tab outer edge 269 to lower stiffness at the tab portion inner boundary 273. For any example of non-homogenous stiffness along either inflow portion 274 and/or tab stiff portions 272, the minimal stiffness will be equal to or greater than the stiffness of the movable body portion 270.

While FIG. 7 illustrates an example of a non-uniform leaflet 262 that includes both a stiffer inflow portion 274 and tab stiff portions 272, which are shown to be separated from each other, in some examples, the inflow portion 274 can extend all the way to, and converge with, the tab stiff portions 272, together forming a continuous stiffer portion of the non-uniform leaflet 262.

FIG. 8 shows an exemplary cross-sectional view of a portion of a non-uniform leaflet 262 coupled to a frame 110. The non-uniform leaflet 262 shown in FIG. 8 includes a stiffer inflow portion 274, which can be coupled to the frame 110 by suturing the inflow portion 274 to struts 114 of the frame 110, such as angled struts 115. The connecting suture 175 can be looped around the strut 114, extending over the outer surface 113 of the frame 110, along the strut outflow edge 131, through the thickness of the inflow portion 274 (at a region closer to the inflow portion proximal end 275), further extending therefrom along an inner surface of the inflow portion 274 (facing the central longitudinal axis L), back through the thickness of the inflow portion 274 (at a region closer to the cusp edge 264), outward along the strut inflow edge 132 toward the outer surface 113 of the frame 110.

Advantageously, the increased stiffness of the inflow portion 274 allows it to better withstand stresses concentrated at the points of suture penetration, such that leaflet 262 can be directly sutured to the frame 110 without requiring any additional intermediary components, such as an inner skirt, thick suture lines, strips and the like. Stated differently, the inflow portion 274 can be attached (e.g., sutured) to the frame 110 such that it directly contacts the inner surface 112, without any intermediate components such as skirts or fabrics disposed therebetween. With this configuration, the number of components in the prosthetic valve is reduced, which reduces assembly time and expense. Nevertheless, in some examples, additional components can be still added, such as a strip 176 of the type described above with respect to FIG. 5, optionally folded over the inflow portion 274.

The strut 114 to which the inflow portion 274 is coupled can define a strut width Wₛ between the strut inflow edge 132 and the strut outflow edge 131. The inflow portion width W_{L} is preferably greater than the strut width Wₛ to align with the portions of the connecting suture 175 extending along both the strut inflow edge 132 and the strut outflow edge 131. Due to the transition between a stiffer inflow portion 274 and a softer movable body portion 270, the inflow bending lines 278 are not formed at or near the level of suture penetration into the leaflet, but rather at or proximate to the inflow portion proximal end 275. Thus, the position of the inflow portion proximal end 275 relative to the level of suture penetration into the inflow portion 274 dictates the distance by which the inflow bending lines 278 are offset away from the suture penetration points. A proximal offsetting width W_{B} is defined between the strut outflow edge 131, which is indicative of the point of suture penetration into the leaflet, and the inflow portion proximal end 275, such that the inflow portion width W_{L} is at least equal to, and in some examples, greater than, the sum of the strut width Wₛ and the proximal offsetting width W_{B} (i.e., W_{L}≥Wₛ+W_{B}).

In some examples, the inflow portion width W_{L} is at least 1.5 times greater than the strut width Wₛ. In some examples, the inflow portion width W_{L} is at least two times greater than the strut width Wₛ. In some examples, the inflow portion width W_{L} is at least three times greater than the strut width Wₛ. In some examples, the inflow portion width W_{L} is at least five times greater than the strut width Wₛ. In some examples, the proximal offsetting width W_{B} is equal to or greater than half the strut width Wₛ. In some examples, the proximal offsetting width W_{B} is equal to or greater than the strut width Wₛ. In some examples, the proximal offsetting width W_{B} is at least twice as great as the strut width Wₛ. In some examples, the proximal offsetting width W_{B} is at least three times as great as the strut width Wₛ. In some examples, the proximal offsetting width W_{B} is at least five times as great as the strut width Wₛ.

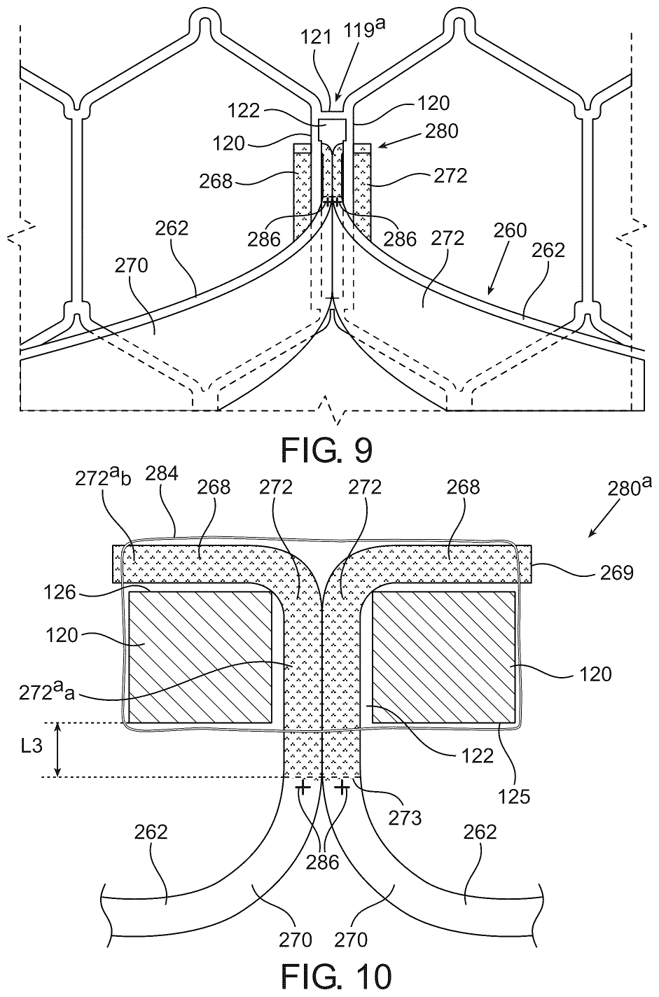

FIG. 9 shows an example of a valvular structure 260 that includes non-uniform leaflets 262 forming a commissure 280 coupled to a commissure window 119 of a prosthetic valve 100. The commissure 280 includes tab stiff portions 272 of adjacent non-uniform leaflets 262, extending through the commissure window opening 122. Outer portions of the tabs 268 can extend through the commissure window opening 122, and fold sideways to extend over the commissure window outer surface 126, which can be also the outer surface 113 of the frame 110 in some examples. The tab stiff portions 272 further extend to some degree radially inward from the commissure window inner surface 125, which can be also the inner surface 112 of the frame 110 in some examples.

In some examples, the tab stiff portions 272 can be semi-rigid, meaning that they are stiffer or more rigid than the movable body portion 270, yet flexible enough to enable them to be bent or folded during commissure assembly. For example, tabs 268 with tab stiff portions 272 can be inserted into a commissure window opening 122 while having a relatively straight configuration, and the portions extending outward can then be bent sideways over the commissure window outer surface 126.

In some example, the tab stiff portions 272 can be relatively rigid, shape-formed to assume a final desired configuration prior to commissure assembly. For example, the tab stiff portions 272 can be placed within a mold or other construct defining their desired shape, and cross-linked in that shape in a manner that will allow them to retain this shape-form during commissure and valve assembly procedures. In some examples, the tab stiff portions 272 are pre-shaped to assume an L-shaped configuration, having one portion radially extending from the free edge 266, and an end portion bent at a straight angle sideways, to rest in parallel of inner or outer surfaces 125, 126 of the commissure window. Such pre-shaped tab stiff portions 272 can advantageously simplify commissure assembly procedures.

In some examples, a commissure 280 that includes tab stiff portions 272 is coupled to a closed commissure window 119^{a}. A closed commissure window 119^{a}, illustrated for example in FIG. 9, is a commissure window completely enclosed along the entire perimeter of the commissure window opening 122, and includes a proximal window bar 121 extending at the proximal end between both window sidewalls 120. While non-rigid or semi-rigid tab stiff portions 272 can be easily passed through a commissure window opening 122 and bent thereafter, pre-shaped tab stiff portions 272, such as L-shaped tab stiff portions 272, can be also utilized with closed commissure window 119^{a}, for example by passing first the outer bent section (also termed second section) of one tab 268 through the commissure window opening 122, and then rotating it such that the outer bent section of one tab 268 resides over the commissure window outer surface 126, followed by the adjacent tab 268 passed and rotated in a similar manner.

FIG. 10 shows a cross-sectional view of one optional configuration by which a commissure 280^{a} is coupled to a commissure window 119, by one or more loops of a suture 284 circumscribing the entire commissure window 119. As shown, each tab stiff portion 272 includes a first section 272^{a}a extending radially through the commissure window opening 122, and a second section 272^{a}b folded sideways, disposed over the commissure window outer surface 126. A suture 284 can extend over the outer sides of the second sections 272^{a}b, through the thickness of the second sections 272^{a}b of the tab stiff portions 272, extending radially inward along the lateral edges of the window sidewalls 120, and then along the commissure window inner surface 125, extending through the thickness of both first sections 272^{a}a of the tab stiff portions 272.

Advantageously, the increased stiffness of the tab stiff portions 272 allows them to better withstand stresses concentrated at the points of suture penetration, such that leaflets 262 can be directly sutured to and/or around a commissure window 119 without requiring any additional intermediary components, such as a commissure coupling member. With this configuration, the number of components in the prosthetic valve is reduced, which reduces assembly time and expense. Nevertheless, in some examples, additional components can be still added, such as a commissure coupling member 182 of the type described above with respect to FIG. 6.

The tabs 268 extending from tab outer edges 268 can generally terminate at the level of the commissure window inner surface 125, while the tab stiff portions 272 further extend radially inward, along the length L3. Due to the transition between the stiffer tab outer edges 268 and the softer movable body portion 270, the commissure bending axes 286 are not formed at or near the level of the commissure window inner surface 125 (or the level of the inner surface 112 of the frame 110), but rather at or proximate to the tab portion inner boundaries 273. Thus, the position of the tab portion inner boundaries 273 relative to the tab portion inner boundaries 273 dictates the distance by which the commissure bending axes 286 are offset away from the commissure window 119 and/or the frame 110.

In some examples, the inner offsetting length L3 is equal to or greater than 500 microns. In some examples, the inner offsetting length L3 is equal to or greater than 1 millimeter. In some examples, the inner offsetting length L3 is equal to or greater than 1.5 millimeters. In some examples, the inner offsetting length L3 is equal to or greater than 2 millimeters.

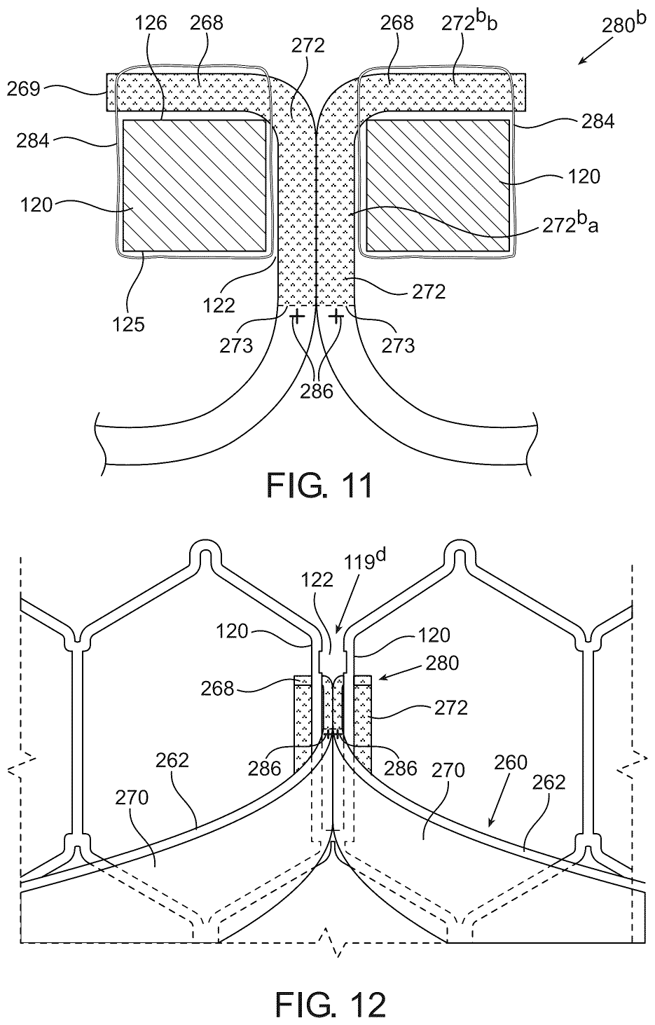

FIG. 11 shows a cross-sectional view of another optional configuration by which a commissure 280^{b} is coupled to a commissure window 119, via separate suture loops coupling each one of the tab stiff portions 272 to a corresponding window sidewall 120. As shown, each tab stiff portion 272 includes a first section 272^{b}a extending radially through the commissure window opening 122, and a second section 272^{b}b folded sideways, disposed over the commissure window outer surface 126. A separate suture 284 can extend over each corresponding outer side of a second section 272^{b}b, through the thickness of the second section 272^{b}b of the tab stiff portion 272, extending radially inward along the lateral edge of the window sidewall 120, and then along the commissure window inner surface 125, toward and through the commissure window opening 122, extending radially outward between the first section 272^{b}a and the window sidewall, and through the thickness of the tab stiff portion 272.

In some examples, the commissure window 119 is an open commissure window, meaning that it is devoid of a proximal window bar (121), leaving the window open ended from above. FIG. 12 shows an example of a valvular structure 260 coupled to an open commissure window 119^{d}. Open commissure windows can be advantageous when the tab stiff portions 272 are pre-shaped, for example to an L-shaped configuration of the type illustrated in FIGS. 10-11, with a second section 272b folded at a substantially right angle with respect to a first section 272a of each tab stiff portion 272. In such cases, the pre-shaped tab stiff portions 272 can be conveniently slid into the open commissure window 119 through the upper open end. Further attachment of the commissure 280 to an open commissure window 119^{d} can be performed according to any other configuration disclosed herein.

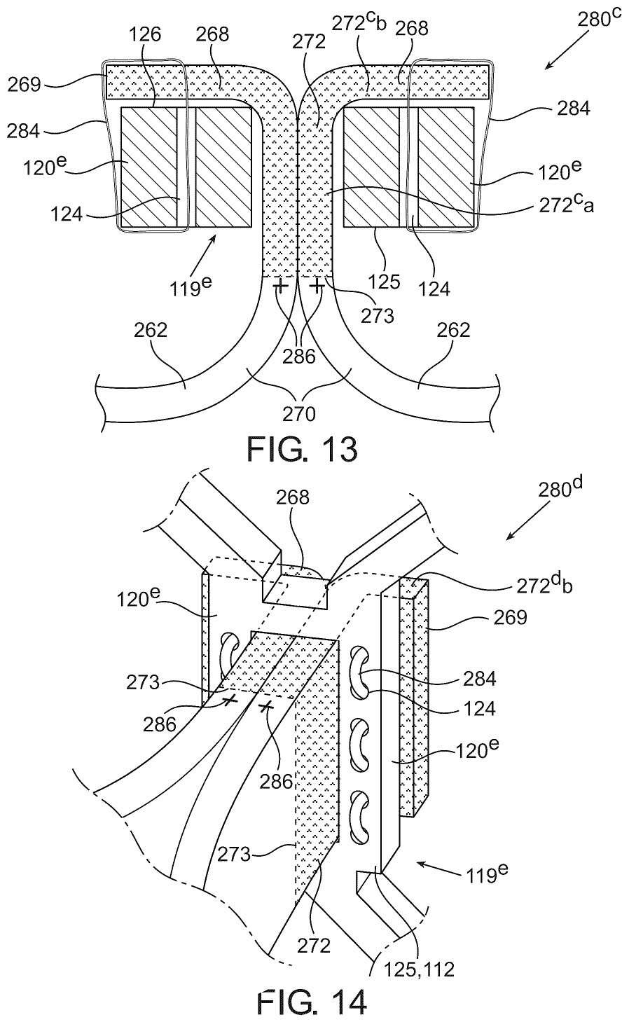

In some examples, a commissure window 119^{e} can include one or more holes 124 extending radially through each of the window sidewalls 120^{e}, through which sutures 284 can be passed. FIG. 13 shows a cross-sectional view of an exemplary configuration by which a commissure 280^{c} can be coupled to a commissure window 119^{e} equipped with window sidewalls 120^{e} having holes extending therethrough. As shown, each tab stiff portion 272 includes a first section 272^{c}a extending radially through the commissure window opening 122, and a second section 272^{c}b folded sideways, disposed over the commissure window outer surface 126. A separate suture 284 can extend over a portion of each corresponding outer side of a second section 272^{c}b, then radially inward around the tab outer edge 269 and along the lateral edge of the window sidewall 120, then along a portion of the commissure window inner surface 125, into and through the hole 124 of the corresponding window sidewall 120, extending further radially outward through the thickness of the second section 272^{c}b of the tab stiff portion 272.

FIG. 14 shows a simplified perspective view of another exemplary configuration by which a commissure 280^{d} can be coupled to a commissure window 119^{e} equipped with window sidewalls 120^{e} having holes extending therethrough, which can differ from the configuration illustrated in FIG. 13 in that the sutures 284 are not looped around the window sidewalls, but rather vertically extend in an in-and-out pattern through subsequent holes 124 of the window sidewalls 120^{e} and the thickness of the second sections 272^{d}b of the tab stiff portion 272 disposed over the commissure window outer surface 126.

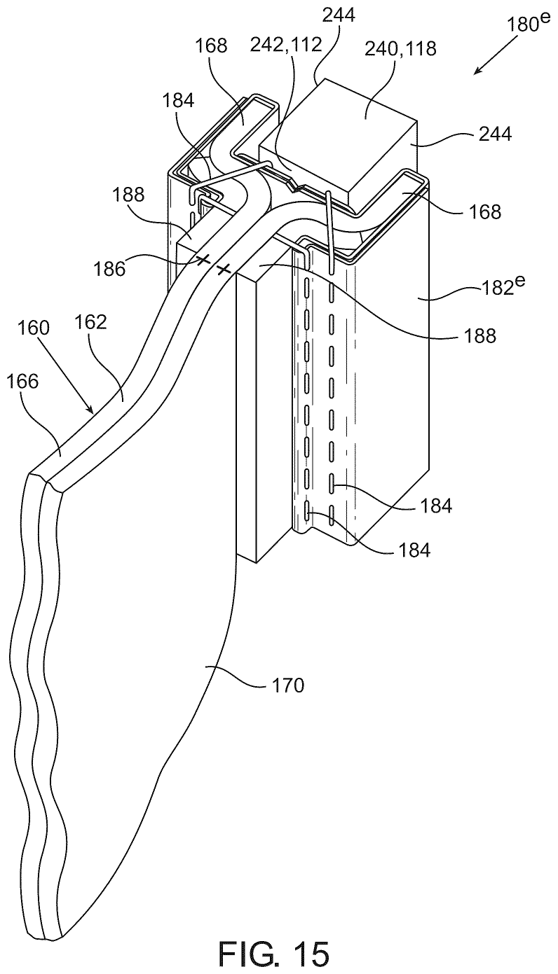

In some examples, commissures of prosthetic valves are coupled to commissure support posts instead of commissure windows. A commissure support post can be any vertically-oriented strut or post member which is devoid of a commissure window, and to which a commissure can be coupled in a variety of appropriate assembly configurations. FIG. 15 shows a simplified perspective view of an exemplary configuration by which a commissure 180^{e} can be coupled to a commissure support post 240. A commissure support post can be a vertical strut integrally formed with the frame of a prosthetic valve, such as a proximal non-windowed axial strut 118. The support post 240 exhibits support post lateral sides 244 and a support post inner surface 242, which can be also the frame's inner surface 112 if the post 240 is integrally formed with the frame. Alternatively or additionally, a commissure support post 240 can be implemented as a vertically oriented post member attached to the frame of a prosthetic valve. For example, certain types of mechanically-expandable prosthetic valve can include actuators attached to the frame, with an upper portion of such actuators further serving as a commissure support post, to which commissures can be coupled.

FIG. 15 illustrates one exemplary configuration of a commissure 180^{e} formed of conventional uniform leaflets 162 can be coupled to such a commissure support post 240. The leaflets 262 can extend radially inward from the support post inner surface 242, with their tabs 168 splayed sideways to extend along the support post inner surface 242, and then radially outward along at least a portion of the support post lateral sides 244, together forming a U-shaped configuration that can be complementary to the inner portion of the commissure support post 240. The commissure 180^{e} can further include reinforcing elements 188 and a commissure coupling member 182^{e} disposed between the commissure support post 240 and the tabs 168, and extending around portions of the tabs 168 and the reinforcing elements 188, with sutures 184 that can extend through various layers of the reinforcing elements 188, tabs 168 and coupling member 182^{e}.

While the reinforcing elements 188 are illustrated in FIG. 15 in the form of strips placed against outer surfaces of the tabs 168, in some examples, the reinforcing members can be implemented as relatively thick sutures, fabrics, tissue patches, and the like. At least a portion of the reinforcing elements 188 extends radially inward relative to the commissure support post 240, increasing the thickness of the inner portion of the commissure 180^{e} in a manner that can offset the commissure bending axes 186 farther away from the support post inner surface 242 and/or the inner surface 112 of the frame. While not explicitly illustrated in FIG. 15, additional sutures can extend through components of the commissure 180^{e} and around the commissure support post 240 to coupled them to each other.

FIG. 16 shows a cross-sectional view of an exemplary configuration by which a commissure 280^{f} comprised of non-uniform leaflets 262 can be coupled to a commissure support post 240. As shown, each tab stiff portion 272 can define an S-shaped configuration, and can be optionally pre-shaped to form the S-shaped configuration, including a first section 272^{f}a extending radially from the tab portion inner boundary 273 toward the commissure support post 240, a second section 272^{f}b folded sideways, disposed over the support post inner surface 242, and a third section 272^{f}c folded again to extend radially outward, along at least a portion of the corresponding support post lateral side 244. A post coupling member 288 can be attached to, and optionally surround, the commissure support post 240, wherein sutures 284 can extend through the thickness of the tab stiff portion 272 and the post coupling member 288 to attached them to each other.

Advantageously, the increased stiffness of the tab stiff portions 272 allows a commissure, such as commissure 280^{f}, to be formed and coupled to a commissure support post 240 without some of the additional components shown if FIG. 15, such as a commissure coupling member. Nevertheless, in some examples, additional components can be still added, such as a commissure coupling member 182^{e} of the type described above with respect to FIG. 15. A further advantage of the proposed configuration is that the tab stiff portions 272, terminating at tab portion inner boundaries 273 which are offset radially inward with respect to the support post inner surface 242 can effectively offset the commissure bending axes 286 radially inward, away from the frame, without the need for added components such as the reinforcing elements (188).

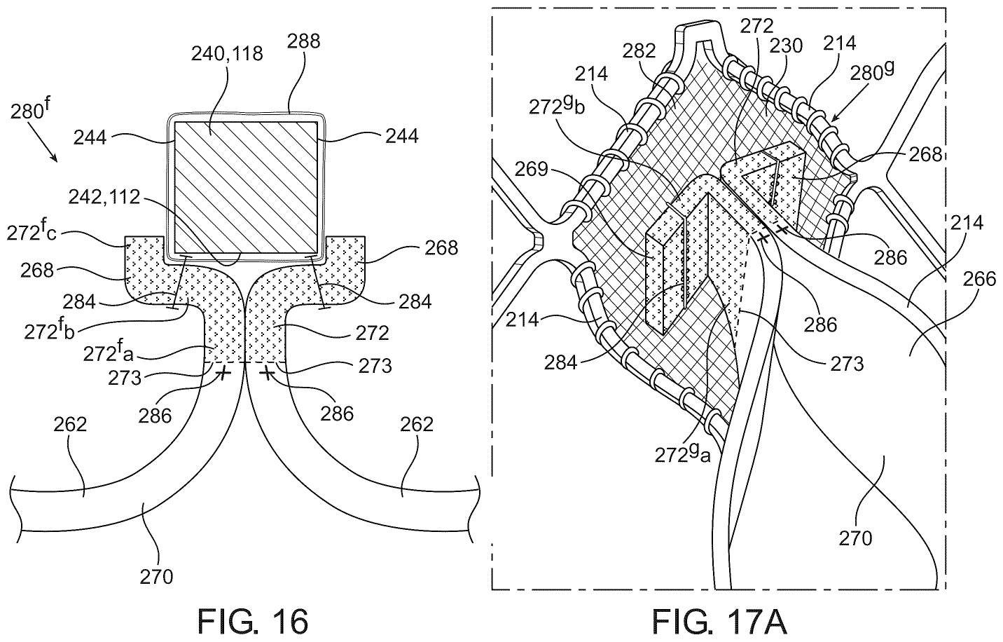

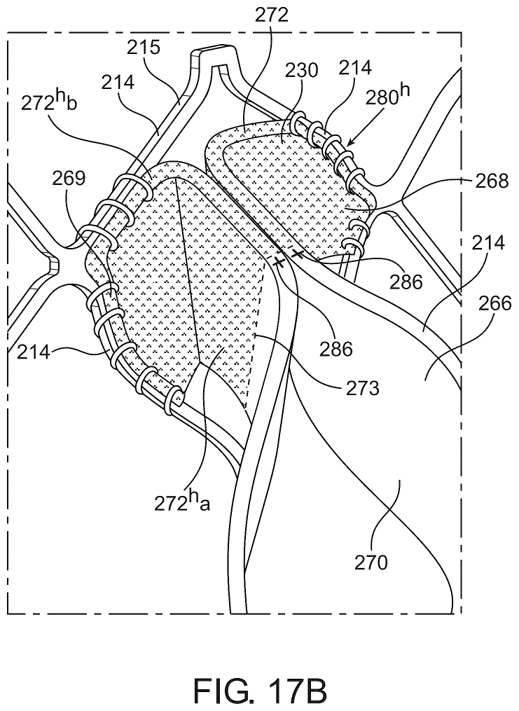

In some examples, a prosthetic valve may lack commissure windows or vertically-oriented commissure support posts. Instead, in such examples, tabs of the commissures can be mounted to cells formed by struts of the frame. FIG. 17A shows a simplified perspective view of an exemplary configuration by which a commissure 280^{g} can be coupled to a cell 230 of a frame. The cell 230 can be formed by a plurality of interconnected struts 214 of the frame, which can be angled struts 215. The cell 230 can be, in some examples, a cell 130 of frame 110, defined by intersecting angled struts 115, or a cell of other types of frame of prosthetic valves. A cell coupling member 282 can extend across the opening of the cells 230 and can be secured to struts 214 forming the cell 230 by one or more sutures. The coupling member 282 can be made from a flexible piece of woven PET fabric, although other synthetic and/or natural materials can be used. In the illustrated example, sutures are shown to extend through the coupling member 282 and loop around the struts 214, around a perimeter of the cell 230.

The tab stiff portions 272 of adjacent non-uniform leaflets 262 can be splayed in opposite directions along the circumferential direction to form a T-shape. The radially-outer surface formed by the splayed tab stiff portions 272 can contact the radially-inner surface of the coupling member 282 and be coupled thereto, for example by one or more sutures 284. In some examples, the tab stiff portions 272^{g} can be pre-shaped to assume L-shaped configurations, defining first sections 272^{g}a extending in a radial direction, and second sections 272^{g}b bent sideways to extend along the inner surface of the coupling member 282, together forming the splayed T-shape when joined. In some examples, the tab stiff portions 272 can be coupled to the coupling member 282 prior to disposing the coupling member 282 within the cell 230 and attaching to struts 214. In some examples, the tab stiff portions 272 can be attached to the coupling member 282 that is already attached to struts 214. In this way, commissure 280^{g} can be mounted to the frame without requiring separate commissure windows or commissure support posts.

Further details regarding mounting of leaflets to valve frames, according to which, valvular structures 260 that include non-uniform leaflets 262 can be similarly coupled to prosthetic valve frames, can be found in U.S. Provisional Application No. 63/024,951, and U.S. Pat. Nos. 9,393,110 and 11,446,614, all of which are incorporated herein by reference in their entireties.

In some examples, a commissure 280 can be directly sutured to struts 216 of a cell 230 without a cell coupling member 282. FIG. 17B shows a simplified perspective view of an exemplary configuration by which a commissure 280^{h}, which can be similar to commissure 280^{g}, can be coupled to a cell 230 of a frame. For example, the second sections 272^{h}b of tab stiff portions 272^{h} can be dimensioned such that in their splayed configuration, they extend across at least a portion of the opening of the cell 230, with tab outer edges 2698 aligned with or extend beyond the struts 214, such as angled struts 215 defining cell 230. The second sections 272^{h}b of tab stiff portions 272^{h} can be directly sutured to such struts, as illustrated in FIG. 17B.

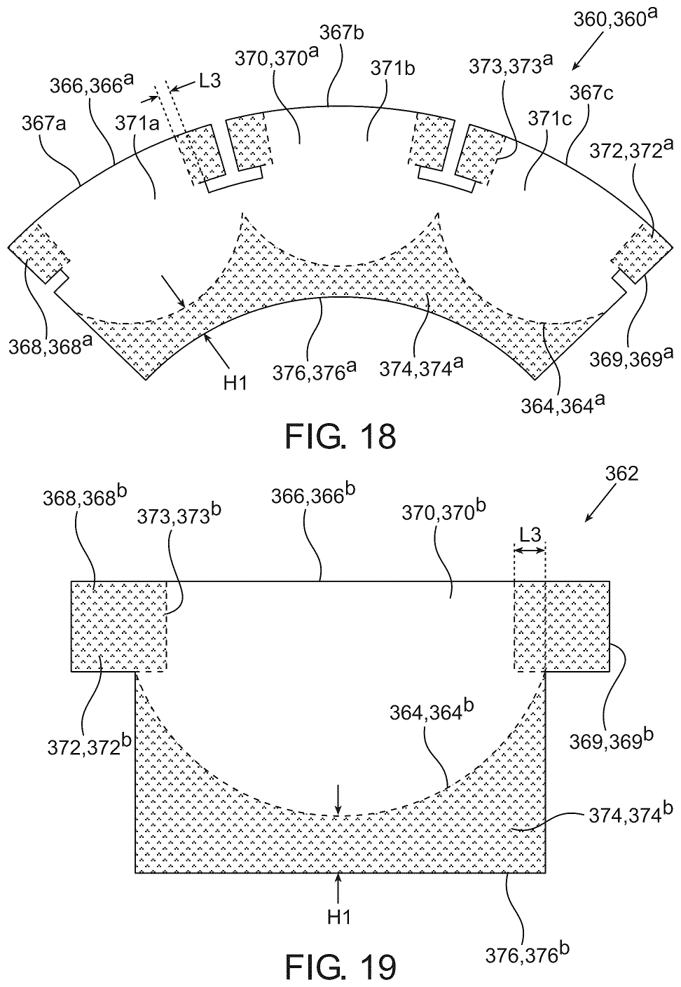

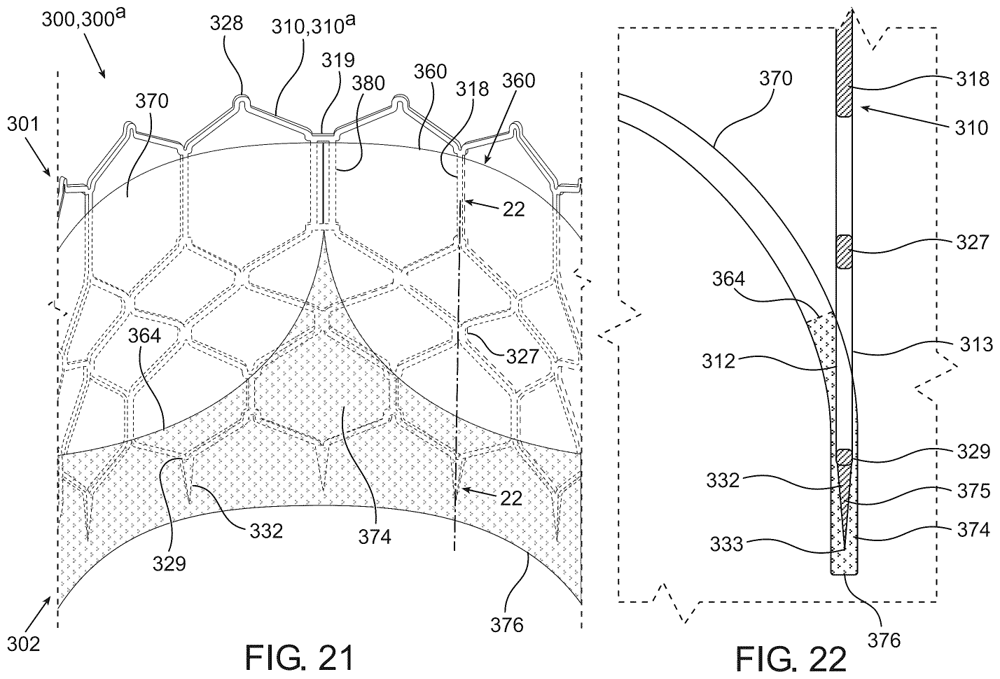

Referring to FIGS. 18-22, a prosthetic valve 300 (shown, for example, in FIG. 21) can include a non-uniform valvular structure 360 (examples of which are shown in FIGS. 18 and 19) coupled to a frame 310. Prosthetic valve 300 comprises an outflow end 301 and an inflow end 302, and can be in some examples, as described for prosthetic valve 100, a balloon expandable valve, though it is to be understood that other valve types are similarly contemplated. Prosthetic valve 300 can be similar to any example described above for prosthetic valve 100, with like numbers referring to like components, except that it further comprises vertical spikes 332 extending distally from the inflow apices 329. The vertical spikes are axially oriented, parallel to the central longitudinal axis L of the prosthetic valve.

Valvular structure 360 includes a movable body portion 370 extending distally from a free edge 366 thereof, and a inflow stiff portion 374 extending proximally from valvular distal edge 376 opposite to the free edge 366. The inflow stiff portion 374 is a portion of the valvular structure 360 through which the vertical spikes 332 extend when the prosthetic valve 300 is assembled. An inflow portion proximal boundary 364 designates the border between the inflow stiff portion 374 and the movable body portion 370 of the valvular structure. The movable body portion 370 is defined between the free edge 366 and the inflow stiff portion 374, for example between the free edge 366 and the inflow portion proximal boundary 364. The movable body portion 370 defines the portion of the valvular structure 360 which is not immovably affixed to the frame 310 when the prosthetic valve 300 is assembled, thus allowed to freely move toward the frame 310 in an open state of the valvular structure 360, and toward central longitudinal axis L in a closed state of the valvular structure 360.

In some examples, movable body portion 370 and inflow stiff portion 374 of a valvular structure 360 constitute different regions of a unitary continuous piece of material. In some examples, the inflow portion proximal boundary 364 comprises a visual marking, such as a visible die-colored marking, a suture extending along the inflow portion proximal boundary 364, and the like, which can help during the assembly procedure to properly distinguish the inflow stiff portion 374 into which the vertical spikes 332 can penetrate.

The valvular structure 360 can further include tabs 368 configured to form commissures as disclosed above. In some examples, the valvular structure 360 further includes tab stiff portions 372 extending along the tabs 368 from the tab outer edges 369 to tab portion inner boundaries 373, wherein the tab stiff portions 372 are stiffer than the movable body portion 370. Each tab portion inner boundary 373 can be a border between the movable body portion 370 and the corresponding tab stiff portion 372, defining a stiff tab length L1 between the tab outer edge 369 and the tab portion inner boundary 373. While only length L3 is indicated in FIGS. 18-19, it is to be understood that the lengths L1, L2 and L3 can be defined in a manner equivalent to that described above with respect to FIG. 7.

The terms “valvular structure 360” and “non-uniform valvular structure 360”, as used herein, are interchangeable, indicating that the material properties of the valvular structure 360 are not homogenous. For example, inflow stiff portion 374 is stiffer than movable body portion 370. While shown to include tab stiff portions 372 in FIGS. 18-19, it is to be understood that the inclusion of tab stiff portions 372 is optional, and in some examples, a non-uniform valvular structure 360 can include a stiffer inflow stiff portion 374, while the tabs 368 can have the same stiffness as that of movable body portion 370.

A unitary piece of material can be provided with non-homogenous material properties. For example, valvular structure 360 can be formed from natural tissue, such as pericardial tissue, undergoing biological treatment procedures that can include subjecting the tissue to cross-linking agents, which can influence its final material properties. In such an example, each of the movable body portion 370 and inflow stiff portion 374 can be subjected to a different biological procedure, including being subjected to different cross-linking agents, subjected to such agents for different time durations, or other procedural variations, adapted to result in an inflow stiff portion hat can be stiffer than the corresponding movable body portion 370. When further including stiffer tab stiff portions 372, they can be treated in a similar manner to that described for inflow stiff portion 374.

The movable body portion 370 can be designed to have material properties substantially similar to those of conventional leaflets 162 described above with respect to FIGS. 1A-3, allowing this portion to move between the closed and open states in a similar manner. The inflow stiff portion 374 is stiffer than the movable body portion 370, facilitating easier penetration of vertical spikes 332 thereinto, and improving engagement retention with the spikes. The inflow stiff portion 374 can be semi-rigid, meaning that it is stiffer or more rigid than the movable body portion 370, yet flexible enough to transition between the crimped and expanded configurations of prosthetic valve 300 without experiencing material failure and without resisting such transitions of the valve.

In some examples, the valvular structure 360 can be formed from two different materials, joined to each other along inflow portion proximal boundary 364 (for example, by suturing, gluing, welding, and the like), wherein the material forming the inflow stiff portion 374 can be stiffer than the material forming the movable body portion 370.

FIG. 18 shows one example of a non-uniform valvular structure 360^{a}, that can be formed of a unitary piece of material designed to cover the entire circumference of prosthetic valve 300. For example, movable body portion 370^{a} can define a plurality of movable regions 371, such as movable regions 371^{a}, 371^{b} and 371^{c} shown in the illustrated configuration. The valvular structure 360^{a} can be cut along an upper region thereof in a manner that separates between adjacent tabs 368^{a}, in which case the free edge 366^{a} can include a corresponding plurality of free edge portions 367, such as three edge portions 367a, 367b and 367c that can be separate from each other, as shown in the illustrated example.

In some examples, a valvular structure 360^{a} can include a unitary movable body portion 370^{a} defining a plurality of movable regions (e.g., regions 371a, 371b, 371c), while the inflow stiff portion 374^{a} can be made of a different (e.g., stiffer) material joined to the movable body portion 370^{a} along inflow portion proximal boundary 364^{a}, or can be made from the same material, continuous with movable body portion 370^{a}, provided with different material properties-such as by being subjected to a different biological treatment procedure.

Since each two adjacent tabs 368^{a} are approximated to each other and joined to form a commissure 380 that can extend radially outward when assembled in the valve 300, the valvular structure 360^{a} can, in some examples, follow a non-linear but rather arcuate profile in a flattened configuration, as shown in FIG. 18, such that both the free edge 366^{a} and the valvular distal edge 376^{a} follow an arcuate path, allowing the valvular structure 360^{a} to assume a final substantially circular configuration when attached to the frame 310. Nevertheless, it is to be understood that in some examples, any of the distal edge 376^{a} and/or free edge 366^{a} can have a relatively linear configurations, optionally resulting in a rectangularly-shaped valvular structure 360^{a} (in its flattened configuration) made of a unitary piece of material.

Another example of a valvular structure 360^{b} can be formed from separate non-uniform leaflets 362 of the type shown in FIG. 19. Each non-uniform leaflet 362 can include a movable body portion 370^{b} extending distally from a free edge 366^{b}, with two opposing tabs 368^{b}. The inflow stiff portion 374^{b} can be made of a different stiffer material joined to the movable body portion 370^{b} along inflow portion proximal boundary 364^{b}, or both can be made from the same material, wherein inflow stiff portion 374^{b} exhibits different material properties—such as by being subjected to a different biological treatment procedure.

In some examples, the inflow portion proximal boundary 364 is non-linear. For example, inflow portion proximal boundary 364^{b} defined for a single leaflet 362 or a portion of inflow portion proximal boundary 364^{a} along one movable region 371 of valvular structure 360^{a}, can generally track a shape similar to an arcuate cusp edge 164 shown for conventional leaflets 162, such that the shape of inflow portion proximal boundary 364 formed by combining three leaflets 362 joined together to form valvular structure 360^{b}, or its shape along the entire circumference of valvular structure 360^{a}, can generally resemble a scalloped line shape, as illustrated. In some examples, the inflow portion proximal boundary 364 is dictated by the shape of a lower or inflow end of frame 310, and thus can follow other patterns, such as substantially zig-zagged paths and the like.

Movable body portion 370 can be dimensioned to generally mimic the shape and size of leaflet body 170 of a conventional leaflet 162 in the case of valvular structure 360^{b}, or a combination of leaflet bodies 170 of a conventional valvular structure 160 in the case of valvular structure 360^{a}, of the types described above with respect to FIGS. 1A-3. A minimal inflow portion height H1 can be defined between the lowermost point along the inflow portion proximal boundary 364, such as a middle point along inflow portion proximal boundary 364, and a vertically opposing point at the valvular distal edge 376, as illustrated in FIGS. 18 and 19.

In some examples, any valvular structure 360 described above, including any of a valvular structure 360^{a} formed of a unitary piece of material or a valvular structure 360^{b} formed of a plurality of separate leaflets 362, can be coupled to a frame 110 according to any of the configurations described above for valvular structures 260 in conjunction with FIGS. 8-17, mutatis mutandis. In some examples, valvular structures 360 can be coupled to a frame 310 as will be described in greater detail below.

The frame 310 comprises a plurality of struts 314 configured in an annular shape, and defines an inner surface 312 facing central axis L and an outer surface 313 facing the opposite direction, wherein surfaces 312, 313 are defined by the struts 314. A frame 310 can comprise, in some examples, a plurality of rows or rungs of angled struts, as well as axial struts that can extend between some rungs of angled struts. The distal-most junctions 327 at which struts 314 of the frame 310 intersect define inflow apices 329, from which vertical spikes 332 can distally extend.

The vertical spikes 332 of a frame 310 have a height, which can be any of spike heights H2 or H3 as will be further elaborated below, terminating with tips 333. In some examples, tips 333 are relatively sharp tips, designed to easily penetrate into an inflow stiff portion 374. The height of the vertical spikes is designed to retain engagement between the frame 310 and the valvular structure 360. For example, all vertical spikes 332 can have spike heights greater than the strut width Ws of angled struts 314 (e.g., of angled struts 315) of the frame 310. If different vertical spikes 332 are provided, having different spike heights, the relationship between the spike height (e.g., height H2 or H3 which will be further described below) and the strut width Ws refers to the minimal value of a spike height of any of the vertical spikes. In some examples, the spike height of any of the vertical spikes is at least two times greater than strut width Ws. In some examples, the spike height of any of the vertical spikes is at least three times greater than strut width Ws. In some examples, the spike height of any of the vertical spikes is at five two times greater than strut width Ws. In some examples, the spike height of any of the vertical spikes is at least ten times greater than strut width Ws.

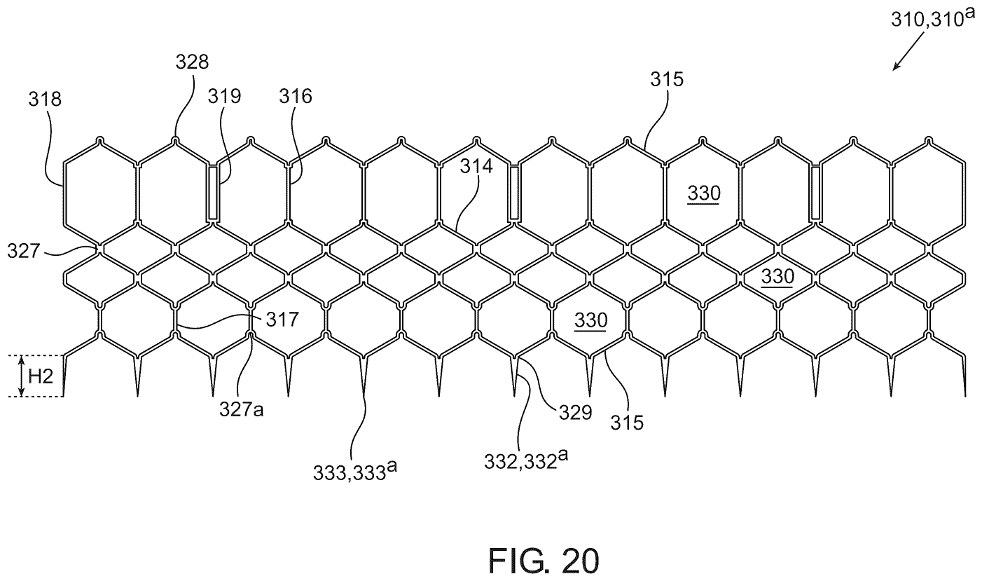

The vertical spikes 332 can be uniformly or non-uniformly shaped, such as having a uniform height for all spikes, or different heights for different groups of spikes. One example of a frame 310^{a} is shown in FIG. 20 in a flat configuration for purposes of illustration. Frame 310^{a} is shown to include a plurality of vertical spikes 332^{a}, all having a similar first spike height H2 defined between the inflow apices 329 and tips 333.

FIG. 21 shows a portion of an exemplary prosthetic valve 300 that includes a non-uniform valvular structure 360 coupled to a frame 310 equipped with a plurality of vertical spikes 332. FIG. 22 shows a partial sectional view across line 22-22 of FIG. 21. As shown, the vertical spikes 332 can penetrate into the thickness of the inflow stiff portion 374 to retain engagement with the valvular structure 360. The vertical spikes 332 can have a tapering thickness beginning with a maximal thickness at their base along the junctions 327 (e.g., along inflow apices 329) they extend from, converging in the distal direction to a narrower tip 333, such as in examples in which the tip 333 is implemented as a sharp tip 333. The maximal thickness of the vertical spikes 332 can be similar to the thickness of struts 314 of the frame 310, measured between the inner surface 312 and the outer surface 313. In some examples, the thickness of the inflow stiff portion 374 is greater than the maximal thickness of the vertical spikes 332, so as to conceal the vertical spikes 332 along their entire length or height within the inflow stiff portion 374. In some examples, the minimal inflow portion height H1 is greater than the first spike height H2, to conceal the sharp tips 333 within the material of the inflow stiff portion 374.

This configuration enables the inflow portion of a leaflet structure to be coupled to the frame without the use of sutures or intermediate components such as an inner skirt, which can advantageously simplify assembly procedure and reduce procedural duration and costs. Moreover, due to the transition between a stiffer inflow stiff portion 374 and a softer movable body portion 370, the inflow bending lines can be offset farther away from the regions of direct engagement with the frame 310 and closer to the inflow portion proximal boundary 364.

In some examples, the tips 333 are not necessarily sharp. For example, a jig or other dedicated tool or apparatus (not shown) can be used to pre-form vertical openings 365 extending in an axial direction (for example, parallel to central axis L when the leaflet structure 360 is coupled to the frame 310), configured to receive and accommodate the respective vertical spikes 332 therein. In such examples, the vertical spikes 332 may slide into the pre-formed vertical openings 365, enabling the tips 333 to optionally be formed as atraumatic end portions instead of sharp tips. Additional coupling members, such as sutures, can be optionally utilized to reinforce attachment between the inflow stiff portion 374 and the frame 310.

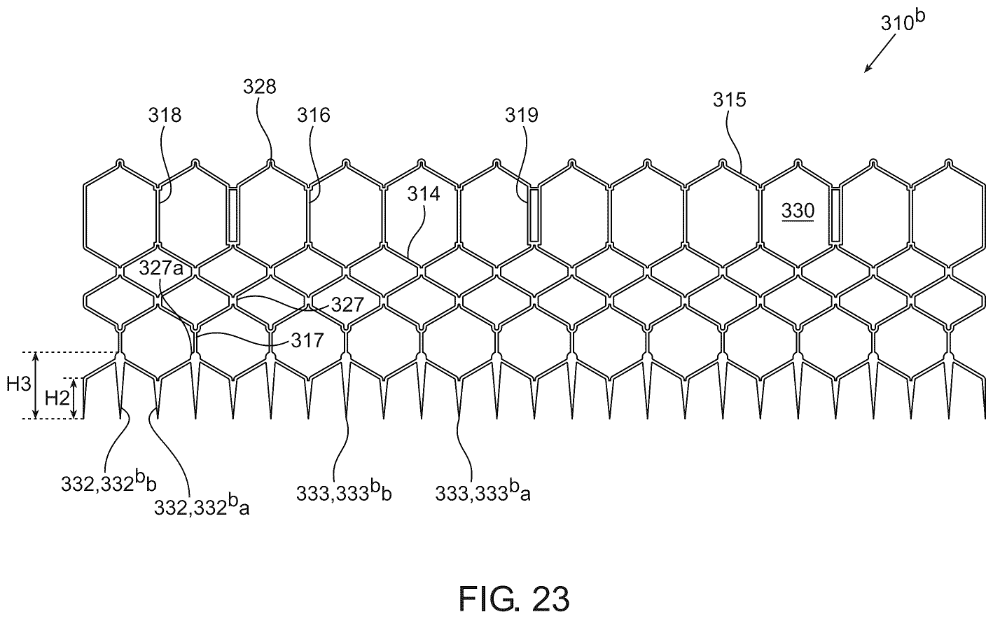

FIG. 23 shows another exemplary frame 310^{b} shown in a flat configuration. Frame 310^{b} can be similar to frame 310^{a}, except that it includes at least two sets of vertical spikes 332, extending from different junction levels along different lengths of heights. In the illustrated example, a first set of vertical spikes 332^{b}a can extend from inflow apices 329 along a first spike height H1, in a similar manner to that shown for vertical spikes 332^{a} in FIG. 20. A second set of spikes 332^{b}b can extend from distal-most non-apical junctions 327a (defined as the junctions 327 closest to inflow apices 329) along a second spike height H3, wherein H3 can be greater than H2. In some examples, the heights H2 and H3 are selected such that the tips 333^{b}a and 333^{b}b of both sets will be axially aligned when the prosthetic valve 300 is in an expanded configuration. The additional set of vertical spikes can improve retention of the inflow stiff portion 374.

As mentioned, any of the valvular structures 360 described above with respect to FIGS. 18 and 19 can include tabs 368 with or without tabs stiff portions 372. When provided with tab stiff portions 374, commissures 380 can be formed and coupled to the frame 310 according to any of the examples described above with respect to FIGS. 9-17, mutatis mutandis.

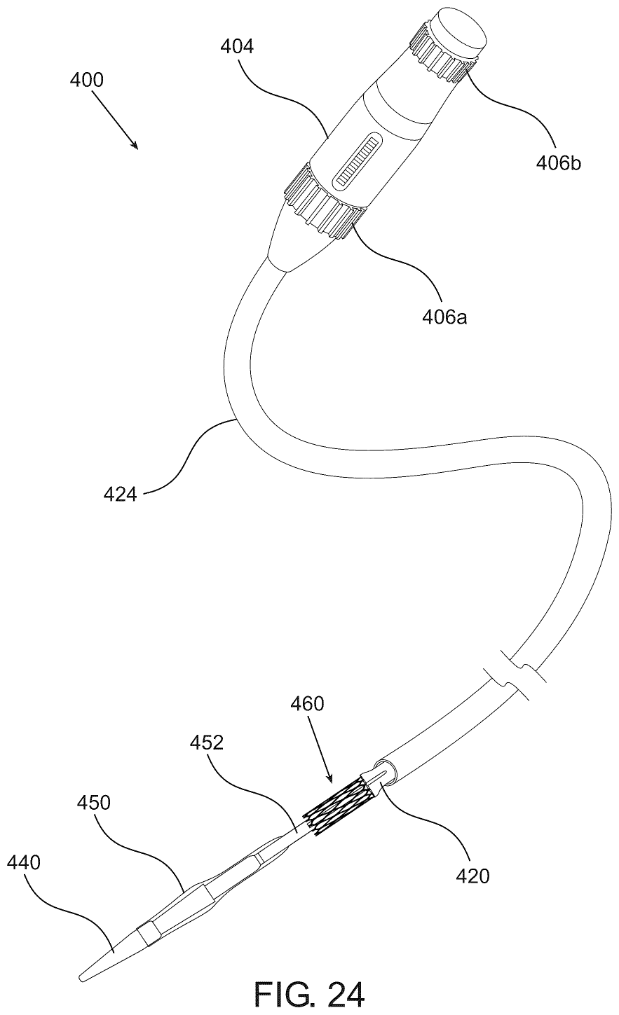

FIG. 24 illustrate a delivery apparatus 400, according to one configuration, adapted to deliver a balloon expandable prosthetic valve 460 described herein (e.g., prosthetic valve 100 or 300). It should be understood that the delivery apparatus 400 can be used to implant prosthetic devices other than prosthetic valves, such as stents or grafts.

The delivery apparatus 400 includes a handle 404 and a balloon catheter 452 having an inflatable balloon 450 mounted on its distal end. The prosthetic valve 460 can be carried in a crimped state over the balloon catheter 452. Optionally, an outer delivery shaft 424 can concentrically extend over the balloon catheter 452, and a push shaft 420 disposed over the balloon catheter 452, optionally between the balloon catheter 452 and the outer delivery shaft 424.

The outer delivery shaft 424, the push shaft 420, and the balloon catheter 452, can be configured to be axially movable relative to each other. For example, a proximally oriented movement of the outer delivery shaft 424 relative to the balloon catheter 452, or a distally oriented movement of the balloon catheter 452 relative to the outer delivery shaft 424, can expose the prosthetic valve 460 from the outer delivery shaft 424. The delivery apparatus 400 can further include a nosecone 440 carried by a nosecone shaft (hidden from view in FIG. 24) extending through a lumen of the balloon catheter 452.

The proximal ends of the balloon catheter 452, the outer delivery shaft 424, the push shaft 420, and optionally the nosecone shaft, can be coupled to the handle 404. During delivery of the prosthetic valve 460, the handle 404 can be maneuvered by an operator (e.g., a clinician or a surgeon) to axially advance or retract components of the delivery apparatus 400, such as the nosecone shaft, the balloon catheter 452, the outer delivery shaft 424, and/or the push shaft 420, through the patient's vasculature, as well as to inflate the balloon 450 mounted on the balloon catheter 452, so as to expand the prosthetic valve 460, and to deflate the balloon 450 and retract the delivery apparatus 400 once the prosthetic valve 460 is mounted in the implantation site.

The handle 404 can include a steering mechanism configured to adjust the curvature of the distal end portion of the delivery apparatus 400. In the illustrated embodiment, for example, the handle 404 includes an adjustment member, such as the illustrated rotatable knob 406a, which in turn is operatively coupled to the proximal end portion of a pull wire. The pull wire can extend distally from the handle 404 through the outer delivery shaft 424 and has a distal end portion affixed to the outer delivery shaft 424 at or near the distal end of the outer delivery shaft 424. Rotating the knob 406a can increase or decrease the tension in the pull wire, thereby adjusting the curvature of the distal end portion of the delivery apparatus 400. Further details on steering or flex mechanisms for the delivery apparatus can be found in U.S. Pat. No. 9,339,384, which is incorporated by reference herein. The handle 404 can further include an adjustment mechanism including an adjustment member, such as the illustrated rotatable knob 406b. The adjustment mechanism can be configured to adjust the axial position of the push shaft 420 relative to the balloon catheter.

The prosthetic valve 460 can be carried by the delivery apparatus 400 during delivery in a crimped state, and expanded by balloon inflation to secure it in a native heart valve annulus. In one exemplary implantation procedure, the prosthetic valve 460 is initially crimped over the balloon catheter 452, proximal to the inflatable balloon 450. Because prosthetic valve 460 is crimped at a location different from the location of balloon 450, prosthetic valve 460 can be crimped to a lower profile than would be possible if it was crimped on top of balloon 450. This lower profile permits the clinician to more easily navigate the delivery apparatus 400 (including crimped prosthetic valve 460) through a patient's vasculature to the treatment location. The lower profile of the crimped prosthetic valve is particularly helpful when navigating through portions of the patient's vasculature which are particularly narrow, such as the iliac artery.

The balloon 450 can be secured to balloon catheter 452 at its balloon proximal end, and to either the balloon catheter 452 or the nosecone 440 at its distal end. The distal end portion of the push shaft 420 is positioned proximal to the outflow end (e.g., outflow end 101 or 301) of the prosthetic valve 460.

When reaching the site of implantation, and prior to balloon inflation, the push shaft 420 is advanced distally, allowing its distal end portion to contact and push against the outflow end of prosthetic valve 460, pushing the valve 460 distally therewith. The distal end of push shaft 420 is dimensioned to engage with the outflow end of the prosthetic valve 460 in a crimped configuration of the valve. In some examples, the distal end portion of the push shaft 420 can be flared radially outward, to terminate at a wider-diameter that can contact the prosthetic valve 460 in its crimped state. Push shaft 420 can then be advanced distally, pushing the prosthetic valve 460 therewith, until the crimped prosthetic valve 460 is disposed around the balloon 450, at which point the balloon 450 can be inflated to radially expand the prosthetic valve 460. Once the prosthetic valve 460 is expanded to its functional diameter within a native annulus, the balloon 450 can be deflated, and the delivery apparatus 400 can be retrieved from the patient's body.

In some examples, the delivery apparatus 400 with the prosthetic valve 460 assembled thereon, can be packaged in a sterile package that can be supplied to end users for storage and eventual use. In some examples, the leaflets of the prosthetic valve (typically made from bovine pericardium tissue or other natural or synthetic tissues) are treated during the manufacturing process so that they are completely or substantially dehydrated and can be stored in a partially or fully crimped state without a hydrating fluid. In this manner, the package containing the prosthetic valve 460 and the delivery apparatus 400, can be free of any liquid. Methods for treating tissue leaflets for dry storage are disclosed in U.S. Pat. Nos. 8,007,992 and 8,357,387, both of which documents are incorporated herein by reference.

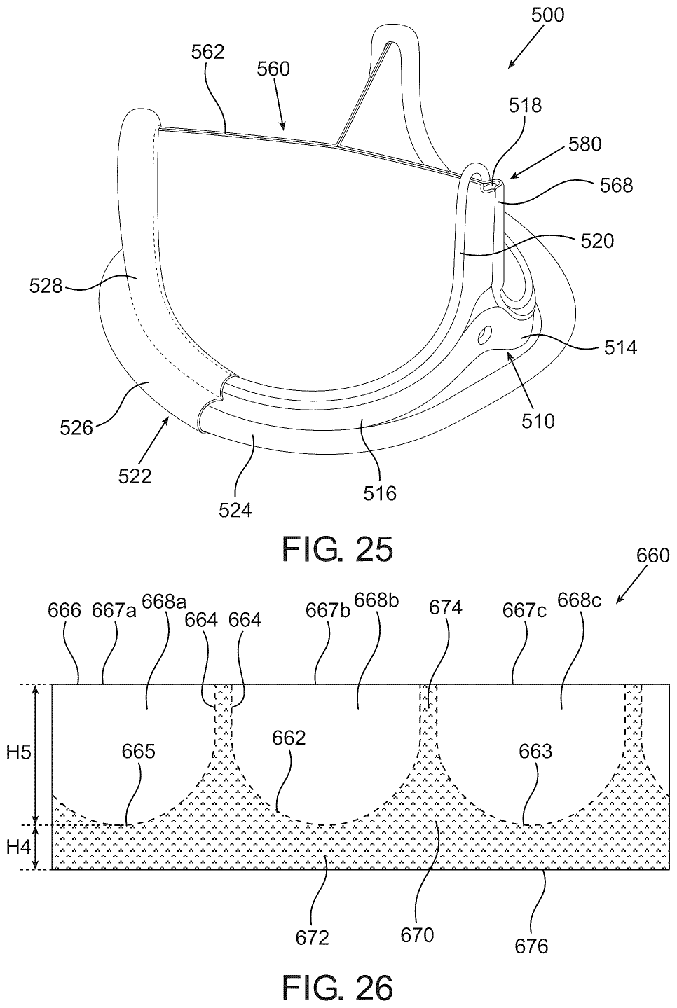

FIG. 25 shows an example of a conventional surgically implantable prosthetic valve 500. In the illustrated example, the prosthetic valve 500 comprises a support frame 510 and a valvular structure 560 attached thereto. The valvular structure 560 comprises a plurality of leaflets 562 (e.g., three leaflets), positioned at least partially within the frame 510, and configured to regulate flow of blood through the prosthetic valve 500. While three leaflets 562 arranged to collapse in a tricuspid arrangement similar to the native aortic valve, are shown in the example illustrated in FIG. 25, it will be clear that a prosthetic valve 500 can include any other number of leaflets 562, such as two leaflets configured to collapse in a bicuspid arrangement similar to the native mitral valve, or more than three leaflets, depending upon the particular application. The leaflets 562 can be generally similar to leaflets 162 described above, and can be made of a flexible material, derived from biological materials (e.g., bovine pericardium or pericardium from other sources), bio-compatible synthetic materials, or other suitable materials as known in the art and described, for example, in U.S. Pat. Nos. 6,730,118, 6,767,362 and 6,908,481, which are incorporated by reference herein.

The frame 510 comprises a band 514 which is generally rigid and/or expansion-resistant in order to maintain the particular shape and diameter of the prosthetic valve 500, and a plurality of vertically oriented commissure posts 518 (e.g., three posts) extending proximally from the band 514 to support the free edges of the leaflets 562. The band 514 can comprise cusp portions 516 extending between the vertically oriented commissure posts 518. The frame 510 can be metallic, plastic, or a combination of the two.

In some configurations, as illustrated, the surgically implantable prosthetic valve 500 further comprises an undulating wireform 520, configured to provide further support to the leaflets 562. The wireform 520 can include a plurality (e.g., three) large radius wireform cusps supporting the cusp regions of the valvular structure 560, while the ends of each pair of adjacent wireform cusps converge somewhat asymptotically to form upstanding wireform commissure portions that terminate in tips, each extending in the opposite direction as the arcuate wireform cusps and having a relatively smaller radius. The cusp portions 516 and the commissure posts 518 can be sized and shaped so as to correspond to the curvature of the wireform 520.

Wireform 520 typically is formed from one or more pieces of wire but also can be formed from other similarly-shaped elongate members. The wireform can also be cut or otherwise formed from tubing or a sheet of material. The wireform can have any of various cross sectional shapes, such as a square, rectangular, circular, or combinations thereof. In some examples, wireform 520 is made of a relatively rigid metal, such as stainless steel or Elgiloy (a Co—Cr—Ni alloy). In some examples, the wireform 520 further comprises a wireform cloth encapsulating it along its length.

Each of the leaflets 562 can be attached along a cusp edge thereof to a corresponding cusp portion 516 of the band 514 and up along adjacent commissure posts 518. Each leaflet 562 can include a pair of oppositely-directed tabs 568, wherein each respective tab 568 can be aligned with a tab 568 of an adjacent leaflet 562 as shown. Each pair of aligned tabs 568 can be inserted between adjacent upstanding wireform portions. The tabs 568 can then be wrapped around a respective commissure posts 518 of the frame 510. The tabs 568 can be sutured or otherwise coupled to each other and/or to the commissure post 518, thereby forming commissures 580 that project in an outflow direction along the longitudinal axis L of the valve. The wireform 520 and the commissure posts 518 provide flexibility to the commissures 580 which helps reduce stress on the bioprosthetic material of the leaflets 562.

A soft sealing or sewing ring 522 circumscribes the frame 510, for example around the band 514, and is typically used to secure the prosthetic valve to a native annulus such as with sutures. The sewing ring 522 comprises a sewing ring insert 524 and a cloth cover 526. The sewing ring insert 524 can be made of a suture permeable material for suturing the prosthetic valve to a native annulus, as known in the art. For example, the sewing ring insert 524 can be made of a silicone-based material, although other suture-permeable materials can be used. The cloth cover 526 can be formed of any biocompatible fabric, such as, for example, polyethylene terephthalate or polyester fabric.

A skirt 528 can completely cover the frame 510, including the band 514 and the commissure posts 518. The wireform 520 is secured to the inner side of the frame 510, wherein the skirt 528 can, in some examples, cover the wireform 520 as well. The sewing ring 522 can be secured to the frame 510 by being stitched to the skirt 528, or via sutures extending through the sewing ring 522 and apertures of the band 514. The skirt 528 can be formed of any biocompatible fabric, such as, for example, polyethylene terephthalate or polyester fabric.

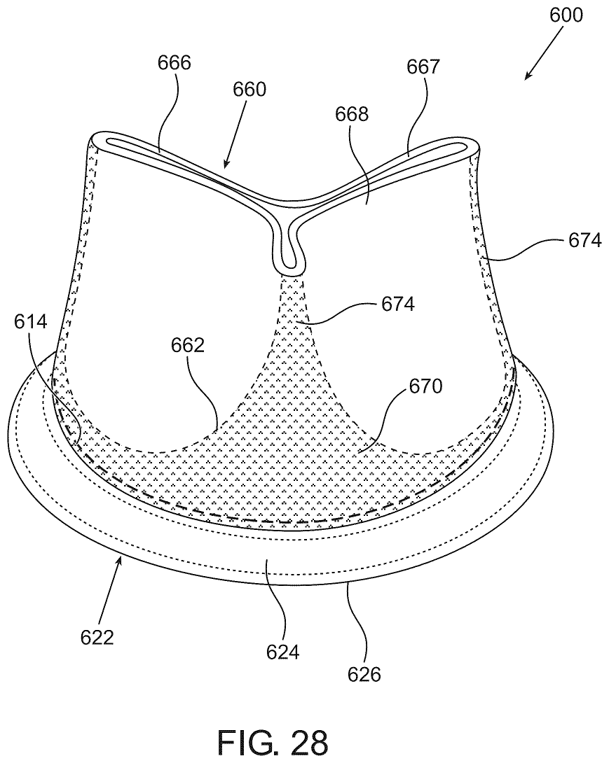

Referring to FIGS. 26-28, a surgically implantable prosthetic valve 600 (shown, for example, in FIG. 28) can include a non-uniform valvular structure 660 coupled to a frame 610 that includes an annular band 614, which can be, in some examples, devoid of vertically oriented commissure posts.

FIG. 26 shows a flattened view of a non-uniform valvular structure 660, that can be formed of a unitary piece of material designed to extend along the entire circumference of prosthetic valve 600. Valvular structure 660 includes a movable regions 668 extending distally from an outflow edge 666 thereof, and a stiff portion 670 extending proximally from valvular distal edge 676 opposite to the outflow edge 666. An undulating boundary 662 designates the border between the stiff portion 670 and the movable regions 668 of the valvular structure. The movable regions 668 are defined between the outflow edge 666 and the stiff portion 670, for example between the outflow edge 666 and the undulating boundary 662. The movable regions 668 and the stiff portion 670 of the valvular structure 660 constitute different regions of a unitary continuous piece of material. The movable regions 668 define the softer portions of the valvular structure 660, allowed to freely move away from each other in an open state of the valvular structure 660, and toward each other and the central longitudinal axis L in a closed state of the valvular structure 660.

The terms “valvular structure 660” and “non-uniform valvular structure 660”, as used herein, are interchangeable, indicating that the material properties of the valvular structure 660 are not homogenous. For example, the stiff portion 670 is stiffer or more rigid than movable regions 668. A unitary piece of material can be provided with non-homogenous material properties. For example, valvular structure 660 can be formed from natural tissue, such as pericardial tissue, undergoing biological treatment procedures that can include subjecting the tissue to cross-linking agents, which can influence its final material properties. In such an example, the stiff portion 670 can be subjected to a different biological procedure than that of the movable regions 668, including being subjected to different cross-linking agents, subjected to such agents for different time durations, or other procedural variations, adapted to result in a stiff portions that can be stiffer or more rigid than the movable regions 668.

The valvular structure 660 can define a plurality of movable regions (e.g., regions 668a, 668b, 668c), while the stiff portion 670 can be continuous with movable regions 668, but provided with different material properties-such as by being subjected to a different biological treatment procedure. The undulating boundary can have boundary cusp-shaped portions 663 and boundary axially-oriented portions 664, such that the cusp-shaped portions 663 are separated from each other, with two boundary axially-oriented portions 664 continuously extending from both sides of each cusp-shaped portion 663, toward and optionally terminating at the outflow edge 666. The axially-oriented portions 664 can be spaced from each other, defining a plurality of stiff post regions 674, which are the regions of the stiff portion 670 bound between adjacent axially-oriented portions 664. The stiff portion can further include a stiff inflow region 672, which is continuous with the stiff post regions 674 and extends therefrom, and from the cusp-shaped portion 663, to the valvular distal edge 676.

The outflow edge 666 can include a plurality of free edge portions, such as free edge portions 667a, 667b and 667c, opposite to corresponding cusp-shaped portions 663, such that each free edge portion 667 extends between axially-oriented portions 664 of a corresponding opposite cusp-shaped portion 663. In some examples, the undulating boundary 662 comprises a visual marking, such as a visible die-colored marking, a suture extending along the undulating boundary 662, and the like, which can help during the assembly procedure to properly distinguish the stiff portion 670 and the movable regions 668.

A minimal stiff portion height H4 can be defined between a distal-most undulating boundary end 665, which can be a middle point along any cusp-shaped portion 663, and a vertically opposing point at the valvular distal edge 676. A maximal movable region height H5 can be similarly defined between the distal-most undulating boundary end 665 and a vertically opposing point at the outflow edge 666. Thus, the sum of H4 and H5 results in a total height of the valvular structure 660 in an unassembled configuration thereof, as shown in FIG. 26. In some examples, H5 is greater than H4. In some examples, H5 is at least two times greater than H4 (i.e., H5>2H4). In some examples, H5 is at least three times greater than H4 (i.e., H5>3H4).

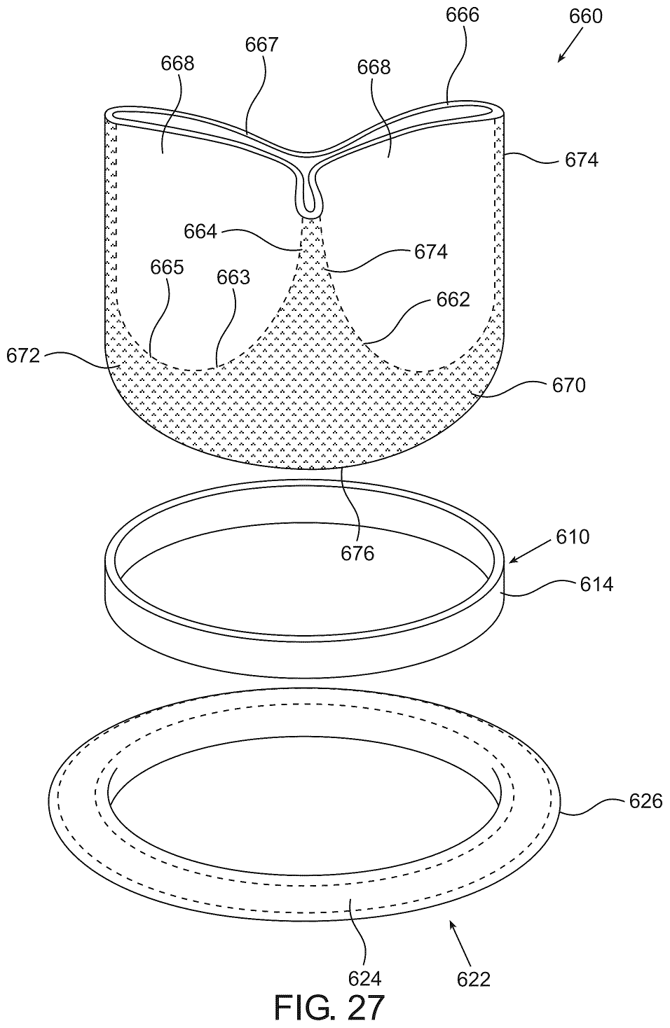

FIGS. 27 and 28 show disassembled and assembled views, respectively, of a surgically implantable prosthetic valve 600. The non-uniform valvular structure 660, and more specifically, its stiff portion 670, can be shape-formed into a desired configuration such that the stiff inflow region 672 assumes an annular configuration, with the stiff post regions 674 extending axially therefrom, is a free state of the valvular structure 660 prior to attachment to the frame 610. For example, a single continuous unit of tissue (e.g., bovine pericardium) can be disposed on or around a mold as part of a process to transition the tissue from an original shape (e.g., flat sheet) to a valve shape. The movable regions 668 can be designed to have material properties substantially similar to those of conventional leaflets 562 of prosthetic valves 500, or leaflets 162 described above with respect to FIGS. 1A-3, allowing this regions to move between the closed and open states in a similar manner. The stiff portion 670 is stiffer than the movable regions 668, and may be rigid or semi-rigid in a manner that allows the stiff post regions 674 to retain a relatively vertically-oriented axial configuration, which can be immovable or slightly bendable radially inward.

The stiffness or rigidity of the stiff post regions 674 can be chosen to adequately support transitioning of the movable regions 668 between the closed and open configurations, without the need for additional support components, such as separate commissure posts or wireforms. Reducing the number of components utilized in an assembly of a prosthetic valve can significantly improve procedural assembly duration and overall costs. Nevertheless, some components, such as commissure posts and/or a wireform can be still used in combination with a non-uniform valvular structure to provide additional stability. In such cases, the assembly efforts can be still significantly improved as there is no need to separately form commissures by joining separate tabs of leaflets in various suturing and folding configurations.

A frame 610 that includes an annular band 614 is illustrated in FIG. 27, without any vertically oriented commissure posts. However, in some examples, as mentioned above, a frame 610 can include both a band 614 and a plurality of commissure posts extending axially therefrom. Moreover, while a ring-shaped annular band 614 is illustrated, in other examples, the band 614 can define non-linear cusp portions. The stiff inflow region 672 can be coupled to the frame 610, such as by being wrapped over the band 614 or otherwise sutured or clamped to the band 614.

A soft sealing or sewing ring 622 can circumscribes the frame 610, for example around the band 614, allowing the prosthetic valve 600 to be secured to a native annulus such as with sutures. The sewing ring 622 can include a sewing ring insert 624 and a cloth cover 626. The sewing ring insert 624 can be made of a suture permeable material for suturing the prosthetic valve to a native annulus, as known in the art. For example, the sewing ring insert 624 can be made of a silicone-based material, although other suture-permeable materials can be used. The cloth cover 626 can be formed of any biocompatible fabric, such as, for example, polyethylene terephthalate or polyester fabric.

While not shows, a skirt, which can be similar to skirt 528 described above with respect to FIG. 25, can cover the band 514. The sewing ring 622 can be secured to the frame 610 by being stitched to the skirt, or via sutures extending through the sewing ring 622 and apertures (not shown) of the band 614. The skirt can be formed of any biocompatible fabric, such as, for example, polyethylene terephthalate or polyester fabric.

### Some Examples of the Disclosed Technology

In view of the above described technology are enumerated below. It should be noted that one feature of an example in isolation or more than one feature of the example taken in combination and, optionally, in combination with one or more features of one or more further examples are further examples also falling within the disclosure of this application.

Example 1. A prosthetic valve, comprising:
a frame movable between a radially compressed state and a radially expanded state;
a valvular structure coupled to the frame and comprising a plurality of non-uniform leaflets configured to regulate flow through the prosthetic valve, wherein each non-uniform leaflet comprises:
a movable body portion disposed between a free edge and an opposite cusp edge; and at least one increased-stiffness portion;
wherein each non-uniform leaflet is formed from a unitary continuous piece of material; and
wherein the at least one increased-stiffness portion is stiffer than the movable body portion.

Example 2. The prosthetic valve of any example herein, particularly example 1, wherein each non-uniform leaflet is formed from natural tissue.

Example 3. The prosthetic valve of any example herein, particularly example 2, wherein each non-uniform leaflet is formed from bovine pericardium.

Example 4. The prosthetic valve of any example herein, particularly any one of examples 1 to 3, wherein the ultimate tensile stress of the increased-stiffness portion is at least 1.5 times greater than the ultimate tensile stress of the movable body portion.

Example 5. The prosthetic valve of any example herein, particularly example 4, wherein the ultimate tensile stress of the increased-stiffness portion is at least 2 times greater than the ultimate tensile stress of the movable body portion.

Example 6. The prosthetic valve of any example herein, particularly any one of examples 1 to 5, wherein the increased-stiffness portion obtains a load at failure which is at least 2 times greater than the load at failure obtained by the movable body portion.

Example 7. The prosthetic valve of any example herein, particularly example 6, wherein the increased-stiffness portion obtains a load at failure which is at least 3 times greater than the load at failure obtained by the movable body portion.

Example 8. The prosthetic valve of any example herein, particularly any one of examples 1 to 7, wherein the plurality of non-uniform leaflets comprises three non-uniform leaflets.

Example 9. The prosthetic valve of any example herein, particularly any one of examples 1 to 8, wherein the at least one increased-stiffness portion comprises an inflow portion extending between the cusp edge and an inflow portion proximal end.

Example 10. The prosthetic valve of any example herein, particularly example 9, wherein the movable body portion extends between the inflow portion proximal end and the free edge.

Example 11. The prosthetic valve of any example herein, particularly example 9 or 10, wherein the inflow portion is attached to the frame.

Example 12. The prosthetic valve of any example herein, particularly example 11, wherein the frame comprises a plurality of intersecting struts, and wherein the inflow portions is coupled to some of the struts of the frame.

Example 13. The prosthetic valve of any example herein, particularly example 12, wherein the inflow portion is sutured to the struts of the frame.

Example 14. The prosthetic valve of any example herein, particularly example 11 or 12, wherein the inflow portion directly contacts an inner surface of the frame.

Example 15. The prosthetic valve of any example herein, particularly example 12, wherein the inflow portion defines an inflow portion width between the cusp edge and the inflow portion proximal end, wherein the struts to which the inflow portion is attached define a strut width between a strut inflow edge and a strut outflow edge, and wherein the inflow portion width is greater than the strut width.

Example 16. The prosthetic valve of any example herein, particularly example 15, wherein the inflow portion width is at least 1.5 times greater than the strut width.

Example 17. The prosthetic valve of any example herein, particularly example 15, wherein the inflow portion width is at least two times greater than the strut width.

Example 18. The prosthetic valve of any example herein, particularly example 15, wherein the inflow portion width is at least three times greater than the strut width.

Example 19. The prosthetic valve of any example herein, particularly example 15, wherein the inflow portion width is at least five times greater than the strut width.

Example 20. The prosthetic valve of any example herein, particularly any one of examples 15 to 19, wherein the inflow portion further defines an offsetting width between the strut outflow edge and the inflow portion proximal end, and wherein the offsetting width is equal to or greater than half the strut width.

Example 21. The prosthetic valve of any example herein, particularly example 20, wherein the offsetting width is equal to or greater than the strut width.

Example 22. The prosthetic valve of any example herein, particularly example 20, wherein the offsetting width is at least twice as great as the strut width.

Example 23. The prosthetic valve of any example herein, particularly example 20, wherein the offsetting width is at least five times as great as the strut width.

Example 24. The prosthetic valve of any example herein, particularly example 20, wherein the offsetting width is at least three times as great as the strut width.

Example 25. The prosthetic valve of any example herein, particularly any one of examples 15 to 24, wherein each non-uniform leaflet further comprises a pair of oppositely-directed tabs between the cusp edge and the free edge.

Example 26. The prosthetic valve of any example herein, particularly example 25, wherein the at least one increased-stiffness portion comprises tab stiff portions, each tab stiff portion extending from a tab outer edge of a corresponding tab to a tab portion inner boundary.

Example 27. The prosthetic valve of any example herein, particularly example 26, wherein the movable body portion extends between the tab portion inner boundaries.

Example 28. The prosthetic valve of any example herein, particularly example 25 or 26, wherein each tab defines a tab length between the tab outer edge and an intersection of the tab with the cusp edge, wherein each tab stiff portion defines a stiff tab length between the tab outer edge and the tab portion inner boundary, and wherein the stiff tab length is greater than the tab length by an inner offsetting length.

Example 29. The prosthetic valve of any example herein, particularly example 28, wherein the offsetting length is equal to or greater than 500 microns.

Example 30. The prosthetic valve of any example herein, particularly example 28, wherein the offsetting length is equal to or greater than 1 millimeter.

Example 31. The prosthetic valve of any example herein, particularly example 28, wherein the offsetting length is equal to or greater than 1.5 millimeters.

Example 32. The prosthetic valve of any example herein, particularly example 28, wherein the offsetting length is equal to or greater than 2 millimeters.

Example 33. The prosthetic valve of any example herein, particularly any one of examples 26 to 32, wherein the tabs of adjacent non-uniform leaflets are joined together to form commissures attached, directly or indirectly, to the frame.

Example 34. The prosthetic valve of any example herein, particularly example 33, wherein the tab stiff portions are pre-shaped to assume a bent configuration in a free state prior to attachment commissure formation.

Example 35. The prosthetic valve of any example herein, particularly example 34, wherein the pre-shaped bent configuration comprises an L-shaped configuration.

Example 36. The prosthetic valve of any example herein, particularly example 34, wherein the pre-shaped bent configuration comprises an S-shaped configuration.

Example 37. The prosthetic valve of any example herein, particularly any one of examples 33 to 36, wherein, when depending on example 28, the tabs stiff portions extend radially inward from the frame along a length which is equal to or greater than the offsetting length.

Example 38. The prosthetic valve of any example herein, particularly example 37, wherein the frame comprises commissure windows, each commissure window comprising a commissure window opening extending between window sidewalls, and wherein each commissure is attached to a corresponding commissure window.

Example 39. The prosthetic valve of any example herein, particularly example 38, wherein each of the tab stiff portion comprises a first section extending radially through a corresponding commissure window opening, and a second section folded sideways over a commissure window outer surface.

Example 40. The prosthetic valve of any example herein, particularly example 39, wherein each commissure is attached to the corresponding commissure window by a suture extending over outer sides of the second sections, through the thickness of the second sections, radially inward along lateral edges of the window sidewalls, along a commissure window inner surface, and through the thickness of the first sections.

Example 41. The prosthetic valve of any example herein, particularly example 39, wherein each commissure is attached to the corresponding commissure window by separate suture loops coupling each one of the tab stiff portions to a corresponding window sidewall.

Example 42. The prosthetic valve of any example herein, particularly example 41, wherein each suture loop extends over a corresponding outer side of the corresponding second section, through the thickness of the second section and along a lateral edge of the corresponding window sidewall, along a commissure window inner surface, toward and through the commissure window opening between the first section and the window sidewall, and through the thickness of the tab stiff portion.

Example 43. The prosthetic valve of any example herein, particularly example 39, wherein each commissure window further comprises holes extending through the window sidewalls.

Example 44. The prosthetic valve of any example herein, particularly example 43, wherein a separate suture extends over a portion of an outer side of each corresponding second section, then radially inward around the tab outer edge and along a lateral edge of the window sidewall, then along a portion of the commissure window outer surface, into and through at least one of the holes of the corresponding window sidewall, and further radially outward through the thickness of the second section.

Example 45. The prosthetic valve of any example herein, particularly example 43, wherein sutures vertically extend in an in-and-out pattern through subsequent holes of the window sidewalls and the thickness of the second sections.

Example 46. The prosthetic valve of any example herein, particularly example 37, wherein the frame comprises commissure support posts, and wherein each commissure is attached to a corresponding commissure support post.

Example 47. The prosthetic valve of any example herein, particularly example 46, wherein each tab stiff portion comprises a first section extending radially from the tab portion inner boundary toward the commissure support post, a second section folded sideways over a support post inner surface, and a third section folded again to extend radially outward along at least a portion of a corresponding support post lateral side.

Example 48. The prosthetic valve of any example herein, particularly example 47, wherein the tab stiff portions are attached via sutures to a coupling member which is attached to the commissure support post.

Example 49. The prosthetic valve of any example herein, particularly example 48, wherein the coupling member surrounds the commissure support post.

Example 50. The prosthetic valve of any example herein, particularly example 37, wherein the prosthetic valve further comprises cell coupling members, each cell coupling member extending across an opening of a cell formed by a plurality of interconnected angled struts of the frame, and wherein each commissure is attached to a corresponding cell coupling member.

Example 51. The prosthetic valve of any example herein, particularly example 50, wherein each cell coupling member is sutured to the angled struts of the corresponding cell.

Example 52. The prosthetic valve of any example herein, particularly example 50 or 51, wherein each cell coupling member is made of a flexible fabric.

Example 53. The prosthetic valve of any example herein, particularly example 52, wherein the flexible fabric comprises a woven PET fabric.

Example 54. The prosthetic valve of any example herein, particularly any one of examples 50 to 53, wherein the tab stiff portions of each commissure are splayed in opposite directions along the circumferential direction to form a T-shape, such that a radially-outer surface formed by the splayed tab stiff portions contacts a radially-inner surface of the corresponding cell coupling member.

Example 55. The prosthetic valve of any example herein, particularly example 37, wherein the tab stiff portions are sutured to interconnected angled struts of the frame defining corresponding cells of the frame.

Example 56. The prosthetic valve of any example herein, particularly example 55, wherein the tab stiff portions of each commissure are splayed in opposite directions along the circumferential direction to form a T-shape, such that a radially-outer surface formed by the splayed tab stiff portions extends across an opening of the corresponding cell.

Example 57. A method of assembling a prosthetic valve, comprising the steps of:
providing a plurality of non-uniform leaflets, each non-uniform leaflet comprising a movable body portion disposed between a free edge and an opposite cusp edge, and at least one increased-stiffness portion; and
attaching the at least one increased-stiffness portion of each non-uniform leaflet to a frame movable between a radially compressed state and a radially expanded state;
wherein each non-uniform leaflet is formed from a unitary continuous piece of material; and
wherein the at least one increased-stiffness portion is stiffer than the movable body portion.

Example 58. The method of any example herein, particularly example 57, wherein each non-uniform leaflet is formed from a unitary continuous piece of material.

Example 59. The method of any example herein, particularly example 58, wherein each non-uniform leaflet is formed from bovine pericardium.

Example 60. The method of any example herein, particularly any one of examples 58 to 59, wherein the ultimate tensile stress of the increased-stiffness portion is at least 1.5 times greater than the ultimate tensile stress of the movable body portion.

Example 61. The method of any example herein, particularly example 60, wherein the ultimate tensile stress of the increased-stiffness portion is at least 2 times greater than the ultimate tensile stress of the movable body portion.

Example 62. The method of any example herein, particularly any one of examples 57 to 61, wherein the increased-stiffness portion obtains a load at failure which is at least 2 times greater than the load at failure obtained by the movable body portion.

Example 63. The method of any example herein, particularly example 62, wherein the increased-stiffness portion obtains a load at failure which is at least 3 times greater than the load at failure obtained by the movable body portion.

Example 64. The method of any example herein, particularly any one of examples 57 to 63, wherein the plurality of non-uniform leaflets comprises three non-uniform leaflets.

Example 65. The method of any example herein, particularly any one of examples 57 to 64, wherein the at least one increased-stiffness portion comprises an inflow portion extending between the cusp edge and an inflow portion proximal end.

Example 66. The method of any example herein, particularly example 65, wherein the movable body portion extends between the inflow portion proximal end and the free edge.

Example 67. The method of any example herein, particularly example 64 or 65, wherein attaching the at least one increased-stiffness portion of each non-uniform leaflet to the frame comprises attaching the inflow portion of each non-uniform leaflet to the frame.

Example 68. The method of any example herein, particularly example 67, wherein attaching the inflow portions to the frame comprises attaching the inflow portions to struts of the frame.

Example 69. The method of any example herein, particularly example 68, wherein attaching the inflow portions to the struts comprises suturing the inflow portions to the struts.

Example 70. The method of any example herein, particularly example 69, wherein suturing the inflow portions to the struts comprises:
extending a connecting suture over an outer surface of the frame;
extending the connecting suture from the outer surface of the frame along a strut outflow edge and through the thickness of the inflow portion;
extending the connecting suture along an inner surface of the inflow portion; and
extending the connecting suture through the thickness of the inflow portion and along a strut inflow edge, toward the outer surface of the frame.

Example 71. The method of any example herein, particularly any one of examples 67 to 70, wherein the inflow portion directly contacts an inner surface of the frame.

Example 72. The method of any example herein, particularly example 68 or 69, wherein the inflow portion defines an inflow portion width between the cusp edge and the inflow portion proximal end, wherein the struts to which the inflow portion is attached define a strut width between a strut inflow edge and a strut outflow edge, and wherein the inflow portion width is greater than the strut width.

Example 73. The method of any example herein, particularly example 72, wherein the inflow portion width is at least 1.5 times greater than the strut width.

Example 74. The method of any example herein, particularly example 72, wherein the inflow portion width is at least two times greater than the strut width.

Example 75. The method of any example herein, particularly example 72, wherein the inflow portion width is at least three times greater than the strut width.

Example 76. The method of any example herein, particularly example 72, wherein the inflow portion width is at least five times greater than the strut width.

Example 77. The method of any example herein, particularly any one of examples 72 to 76, wherein the inflow portion further defines an offsetting width between the strut outflow edge and the inflow portion proximal end, and wherein the offsetting width is equal to or greater than half the strut width.

Example 78. The method of any example herein, particularly example 77, wherein the offsetting width is equal to or greater than the strut width.

Example 79. The method of any example herein, particularly example 77, wherein the offsetting width is at least twice as great as the strut width.

Example 80. The method of any example herein, particularly example 77, wherein the offsetting width is at least five times as great as the strut width.

Example 81. The method of any example herein, particularly example 77, wherein the offsetting width is at least three times as great as the strut width.

Example 82. The method of any example herein, particularly any one of examples 72 to 81, wherein each non-uniform leaflet further comprises a pair of oppositely-directed tabs between the cusp edge and the free edge.

Example 83. The method of any example herein, particularly example 82, wherein the at least one increased-stiffness portion comprises tab stiff portions, each tab stiff portion extending from a tab outer edge of a corresponding tab to a tab portion inner boundary.

Example 84. The method of any example herein, particularly example 83, wherein the movable body portion extends between the tab portion inner boundaries.

Example 85. The method of any example herein, particularly example 83 or 84, wherein each tab defines a tab length between the tab outer edge and an intersection of the tab with the cusp edge, wherein each tab stiff portion defines a stiff tab length between the tab outer edge and the tab portion inner boundary, and wherein the stiff tab length is greater than the tab length by an inner offsetting length.

Example 86. The method of any example herein, particularly example 85, wherein the offsetting length is equal to or greater than 500 microns.

Example 87. The method of any example herein, particularly example 86, wherein the offsetting length is equal to or greater than 1 millimeter.

Example 88. The method of any example herein, particularly example 85, wherein the offsetting length is equal to or greater than 1.5 millimeters.

Example 89. The method of any example herein, particularly example 85, wherein the offsetting length is equal to or greater than 2 millimeters.

Example 90. The method of any example herein, particularly any one of examples 83 to 89, wherein attaching the at least one increased-stiffness portion of each non-uniform leaflet to the frame comprises:
joining the tabs of adjacent non-uniform leaflets together to form commissures; and
attaching the commissures to the frame.

Example 91. The method of any example herein, particularly example 90, further comprising pre-shaping the tab stiff portions to assume a bent configuration in a free state prior to attaching the commissures to the frame.

Example 92. The method of any example herein, particularly example 90, wherein the pre-shaped bent configuration comprises an L-shaped configuration.

Example 93. The method of any example herein, particularly example 90, wherein the pre-shaped bent configuration comprises an S-shaped configuration.

Example 94. The method of any example herein, particularly any one of examples 90 to 93, wherein, when depending on example 85, attaching the commissures to the frame is performed such that the tabs stiff portions extend radially inward from the frame along a length which is equal to or greater than the offsetting length.

Example 95. The method of any example herein, particularly example 94, wherein the frame comprises commissure windows, each commissure window comprising a commissure window opening extending between window sidewalls, and wherein attaching the commissures to the frame comprises attaching each commissure to a corresponding commissure window.

Example 96. The method of any example herein, particularly example 95, wherein each of the tab stiff portion comprises a first section extending radially through a corresponding commissure window opening, and a second section folded sideways over a commissure window outer surface.

Example 97. The method of any example herein, particularly example 96, wherein the commissure window is an open commissure window, wherein attaching the commissure to the commissure window comprises sliding the tab stiff portions into the commissure window opening through an upper open end of the commissure window.

Example 98. The method of any example herein, particularly example 96, wherein the commissure window is closed commissure window comprising a proximal window bar.

Example 99. The method of any example herein, particularly example 98, wherein attaching the commissure to the commissure window comprises:

passing the tab stiff portions through the commissure window opening; and bending the tab stiff portions over the window sidewalls to form the folded second sections.

Example 100. The method of any example herein, particularly example 98, wherein, when depending on example 91 or 92, attaching the commissure to the commissure window comprises:
passing the bent section of a first one of the tabs stiff portions through the commissure window opening;
rotating the first one of the tabs stiff portions in a first direction such that its bent section resides over the commissure window outer surface;
passing the bent section of a second one of the tabs stiff portions through the commissure window opening; and
rotating the second one of the tabs stiff portions in a second direction such that its bent section resides over the commissure window outer surface.

Example 101. The method of any example herein, particularly any one of examples 96 to 100, wherein attaching each commissure to the corresponding commissure window comprises:
extending a suture over outer sides of the second sections;
extending the suture through the thickness of the second sections, and radially inward along lateral edges of the window sidewalls;
extending the suture along a commissure window inner surface; and
extending the suture through the thickness of the first sections.

Example 102. The method of any example herein, particularly any one of examples 96 to 100, wherein attaching each commissure to the corresponding commissure window comprises coupling each of the tab stiff portions to a corresponding window sidewall by separate suture loops.

Example 103. The method of any example herein, particularly example 102, wherein coupling by each suture loops comprises:
extending the suture loop over a corresponding outer side of the corresponding second section;
extending the suture loop through the thickness of the second section and along a lateral edge of the corresponding window sidewall;
extending the suture loop along a commissure window inner surface;
extending the suture loop through the commissure window opening between the first section and the window sidewall; and
extending the suture loop through the thickness of the tab stiff portion.

Example 104. The method of any example herein, particularly any one of examples 96 to 100, wherein each commissure window further comprises holes extending through the window sidewalls.

Example 105. The method of any example herein, particularly example 104, wherein attaching each commissure to the corresponding commissure window comprises coupling each of the tab stiff portions to a corresponding window sidewall by separate sutures.

Example 106. The method of any example herein, particularly example 105, wherein coupling each of the tab stiff portion by each separate suture comprises:
extending the suture over a portion of an outer side of the corresponding second section;
extending the suture around the tab outer edge and along a lateral edge of the window sidewall;
extending the suture along a portion of the commissure window outer surface; and
extending the suture into and through at least one of the holes of the corresponding window sidewall and the thickness of the corresponding second section.

Example 107. The method of any example herein, particularly example 104, wherein attaching each commissure to the corresponding commissure window comprises vertically extending sutures in an in-and-out pattern through subsequent holes of the window sidewalls and the thickness of the second sections.

Example 108. The method of any example herein, particularly example 94, wherein the frame comprises commissure support posts, and wherein attaching the commissures to the frame comprises attaching each commissure to a corresponding commissure support post.

Example 109. The method of any example herein, particularly example 108, wherein attaching each commissure to a corresponding commissure support post comprises:
radially extending a first section of each tab stiff portion from the tab portion inner boundary toward the commissure support post;
folding the tab stiff portions sideways over a support post inner surface; and
folding the tab stiff portions radially outward to extend along at least a portion of a corresponding support post lateral side.

Example 110. The method of any example herein, particularly example 109, wherein attaching each commissure further comprises:

attaching a coupling member to the commissure support post; and suturing the tab stiff portions to the coupling member.

Example 111. The method of any example herein, particularly example 110, wherein attaching a coupling member to the commissure support post comprises wrapping the coupling member around the commissure support post.

Example 112. The method of any example herein, particularly example 94, wherein the frame comprises cells formed by interconnected angled struts of the frame, and wherein attaching the commissures to the frame comprises:
extending cell coupling members across opening of some of the cells; and
attaching each commissure to a corresponding cell coupling member.

Example 113. The method of any example herein, particularly example 112, wherein extending each cell coupling member over the opening of the corresponding cells comprises suturing the cell coupling members to the angled struts of the corresponding cell.

Example 114. The method of any example herein, particularly example 112 or 113, wherein each cell coupling member is made of a flexible fabric.

Example 115. The method of any example herein, particularly example 114, wherein the flexible fabric comprises a woven PET fabric.

Example 116. The method of any example herein, particularly any one of examples 112 to 115, wherein attaching each commissure to a corresponding cell coupling member comprises:
splaying the tab stiff portions in opposite direction along the circumferential direction to form a T-shape;
bringing a radially-outer surface formed by the splayed tab stiff portions in contact with a radially-inner surface of the corresponding cell coupling member; and suturing the splayed tab stiff portions to the cell coupling member.

Example 117. The method of any example herein, particularly example 94, wherein the frame comprises cells formed by interconnected angled struts of the frame, and wherein attaching the commissures to the frame comprises suturing angled struts defining corresponding ones of the cells.

Example 118. The method of any example herein, particularly example 117, wherein attaching each commissure to a corresponding cell further comprises splaying the tab stiff portions in opposite direction along the circumferential direction to form a T-shape, such that a radially-outer surface formed by the splayed tab stiff portions extends across an opening of the corresponding cell, and wherein suturing the tab stiff portion comprises suturing the splayed tab stiff portions to the corresponding angled struts.

Example 119. A prosthetic valve, comprising:
a frame movable between a radially compressed state and a radially expanded state, the frame comprising a plurality of vertical spikes;
a non-uniform valvular structure coupled to the frame and configured to regulate flow through the prosthetic valve, the non-uniform valvular structure comprising:
an inflow stiff portion disposed between a valvular distal edge and an inflow portion proximal boundary; and
a movable body portion disposed between the inflow portion proximal boundary a free edge;
wherein the inflow stiff portion is stiffer than the movable body portion; and
wherein the vertical spikes extend through the inflow stiff portion.

Example 120. The prosthetic valve of any example herein, particularly example 119, wherein at least some of the vertical spikes distally extend from inflow apices of the frame.

Example 121. The prosthetic valve of any example herein, particularly example 119 or 120, wherein the vertical spikes comprise sharp tips which are concealed within the inflow stiff portion.

Example 122. The prosthetic valve of any example herein, particularly example 119 or 120, wherein the vertical spikes comprise atraumatic tips.

Example 123. The prosthetic valve of any example herein, particularly any one of examples 119 to 122, wherein the inflow stiff portions comprises a plurality of pre-formed vertical openings, configured to accommodate the vertical spikes therein.

Example 124. The prosthetic valve of any example herein, particularly any one of examples 119 to 123, wherein the non-uniform valvular structure is formed from natural tissue.

Example 125. The prosthetic valve of any example herein, particularly example 124, wherein the non-uniform valvular structure is formed from bovine pericardium.

Example 126. The prosthetic valve of any example herein, particularly any one of examples 119 to 125, wherein the ultimate tensile stress of the inflow stiff portion is at least 1.5 times greater than the ultimate tensile stress of the movable body portion.

Example 127. The prosthetic valve of any example herein, particularly example 126, wherein the ultimate tensile stress of the inflow stiff portion is at least 2 times greater than the ultimate tensile stress of the movable body portion.

Example 128. The prosthetic valve of any example herein, particularly any one of examples 119 to 127, wherein the inflow stiff portion obtains a load at failure which is at least 2 times greater than the load at failure obtained by the movable body portion.

Example 129. The prosthetic valve of any example herein, particularly example 128, wherein the inflow stiff portion obtains a load at failure which is at least 3 times greater than the load at failure obtained by the movable body portion.

Example 130. The prosthetic valve of any example herein, particularly any one of examples 119 to 129, wherein the inflow portion proximal boundary is non-linearly shaped.

Example 131. The prosthetic valve of any example herein, particularly any one of examples 119 to 130, wherein the vertical spikes define a spike height which is greater than a strut width of struts of the frame.

Example 132. The prosthetic valve of any example herein, particularly example 131, wherein the spike height is at least two times greater than the strut width.

Example 133. The prosthetic valve of any example herein, particularly example 131, wherein the spike height is at least three times greater than the strut width.

Example 134. The prosthetic valve of any example herein, particularly example 131, wherein the spike height is at least five times greater than the strut width.

Example 135. The prosthetic valve of any example herein, particularly example 131, wherein the spike height is at least ten times greater than the strut width.

Example 136. The prosthetic valve of any example herein, particularly any one of examples 131 to 135, wherein the inflow stiff portion defines a minimal inflow portion height which is greater than the spike height.

Example 137. The prosthetic valve of any example herein, particularly example 120, wherein the vertical spikes comprise a first set of vertical spikes extending distally from the inflow apices, and a second set of vertical spikes extending distally from distal-most non-apical junctions of the frame.

Example 138. The prosthetic valve of any example herein, particularly example 137, wherein the vertical spikes of the first set define a first spike height, and wherein the vertical spikes of the second set define a second spike height which is different from the first spike height.

Example 139. The prosthetic valve of any example herein, particularly example 138, wherein the inflow stiff portion defines a minimal inflow portion height which is greater than the first spike height.

Example 140. The prosthetic valve of any example herein, particularly example 138 or 139, wherein the second spike height is greater than the first spike height.

Example 141. The prosthetic valve of any example herein, particularly example 140, wherein tips of the first set of vertical spikes are axially aligned with tips of the second set of vertical spikes in the radially expanded state.

Example 142. The prosthetic valve of any example herein, particularly any one of examples 119 to 141, wherein the non-uniform valvular structure is formed of a unitary piece of material designed to cover the entire circumference of the prosthetic valve.

Example 143. The prosthetic valve of any example herein, particularly example 142, wherein the movable body portion comprises a plurality of movable regions.

Example 144. The prosthetic valve of any example herein, particularly example 143, wherein the plurality of movable regions comprises three movable regions.

Example 145. The prosthetic valve of any example herein, particularly any one of examples 142 to 144, wherein the non-uniform valvular structure further comprises two oppositely-directed tabs extending from each movable region, wherein adjacent tabs of each two of the movable regions are joined together to form a commissure, and wherein the commissures are coupled to the frame.

Example 146. The prosthetic valve of any example herein, particularly example 145, wherein the non-uniform valvular structure further comprises tab stiff portions, each tab stiff portion extending from a tab outer edge of a corresponding tab to a tab portion inner boundary, and wherein the tab stiff portions are stiffer than the movable body portion.

Example 147. The prosthetic valve of any example herein, particularly example 146, wherein each movable region extends between two of the tab portion inner boundaries.

Example 148. The prosthetic valve of any example herein, particularly any one of examples 119 to 141, wherein the non-uniform valvular structure comprises a plurality of non-uniform leaflets.

Example 150. The prosthetic valve of any example herein, particularly example 148 or 149, wherein each non-uniform leaflet comprises two oppositely-directed tabs, wherein the tabs of adjacent non-uniform leaflets are joined together to form a commissure, and wherein the commissures are coupled to the frame.

Example 151. The prosthetic valve of any example herein, particularly example 150, wherein the non-uniform leaflets further comprise tab stiff portions, each tab stiff portion extending from a tab outer edge of a corresponding tab to a tab portion inner boundary, and wherein the tab stiff portions are stiffer than the movable body portion.

Example 152. The prosthetic valve of any example herein, particularly example 149, wherein the movable body portion of each non-uniform leaflet extends between the corresponding non-uniform leaflet's tab portion inner boundaries.

Example 153. The prosthetic valve of any example herein, particularly example 146 or 151, wherein the ultimate tensile stress of the tab stiff portions is at least 1.5 times greater than the ultimate tensile stress of the movable body portion.

Example 154. The prosthetic valve of any example herein, particularly example 153, wherein the ultimate tensile stress of the tab stiff portions is at least 2 times greater than the ultimate tensile stress of the movable body portion.

Example 155. The prosthetic valve of any example herein, particularly any one of examples 146-147 or 151-154, wherein the tab stiff portions obtain a load at failure which is at least 2 times greater than the load at failure obtained by the movable body portion.

Example 156. The prosthetic valve of any example herein, particularly example 155, wherein the tab stiff portions obtain a load at failure which is at least 3 times greater than the load at failure obtained by the movable body portion.

Example 157. The prosthetic valve of any example herein, particularly any one of examples 146-147 or 151-156, wherein the tab stiff portions are pre-shaped to assume a bent configuration in a free state prior to attachment commissure formation.

Example 158. The prosthetic valve of any example herein, particularly example 157, wherein the pre-shaped bent configuration comprises an L-shaped configuration.

Example 159. The prosthetic valve of any example herein, particularly example 157, wherein the pre-shaped bent configuration comprises an S-shaped configuration.

Example 160. The prosthetic valve of any example herein, particularly any one of examples 146-147 or 151-159, wherein the tab stiff portions extend along an offsetting length thereof radially inward from the frame.

Example 161. The prosthetic valve of any example herein, particularly example 160, wherein the frame comprises commissure windows, each commissure window comprising a commissure window opening extending between window sidewalls, and wherein each commissure is attached to a corresponding commissure window.

Example 162. The prosthetic valve of any example herein, particularly example 161, wherein each of the tab stiff portion comprises a first section extending radially through a corresponding commissure window opening, and a second section folded sideways over a commissure window outer surface.

Example 163. The prosthetic valve of any example herein, particularly example 162, wherein each commissure is attached to the corresponding commissure window by a suture extending over outer sides of the second sections, through the thickness of the second sections, radially inward along lateral edges of the window sidewalls, along a commissure window inner surface, and through the thickness of the first sections.

Example 164. The prosthetic valve of any example herein, particularly example 162, wherein each commissure is attached to the corresponding commissure window by separate suture loops coupling each one of the tab stiff portions to a corresponding window sidewall.

Example 165. The prosthetic valve of any example herein, particularly example 164, wherein each suture loop extends over a corresponding outer side of the corresponding second section, through the thickness of the second section and along a lateral edge of the corresponding window sidewall, along a commissure window inner surface, toward and through the commissure window opening between the first section and the window sidewall, and through the thickness of the tab stiff portion.

Example 166. The prosthetic valve of any example herein, particularly example 162, wherein each commissure window further comprises holes extending through the window sidewalls.

Example 167. The prosthetic valve of any example herein, particularly example 166, wherein a separate suture extends over a portion of an outer side of each corresponding second section, then radially inward around the tab outer edge and along a lateral edge of the window sidewall, then along a portion of the commissure window outer surface, into and through at least one of the holes of the corresponding window sidewall, and further radially outward through the thickness of the second section.

Example 168. The prosthetic valve of any example herein, particularly example 166, wherein sutures vertically extend in an in-and-out pattern through subsequent holes of the window sidewalls and the thickness of the second sections.

Example 169. The prosthetic valve of any example herein, particularly example 160, wherein the frame comprises commissure support posts, and wherein each commissure is attached to a corresponding commissure support post.

Example 170. The prosthetic valve of any example herein, particularly example 169, wherein each tab stiff portion comprises a first section extending radially from the tab portion inner boundary toward the commissure support post, a second section folded sideways over a support post inner surface, and a third section folded again to extend radially outward along at least a portion of a corresponding support post lateral side.

Example 171. The prosthetic valve of any example herein, particularly example 170, wherein the tab stiff portions are attached via sutures to a coupling member which is attached to the commissure support post.

Example 172. The prosthetic valve of any example herein, particularly example 171, wherein the coupling member surrounds the commissure support post.

Example 173. The prosthetic valve of any example herein, particularly example 160, wherein the prosthetic valve further comprises cell coupling members, each cell coupling member extending across an opening of a cell formed by a plurality of interconnected angled struts of the frame, and wherein each commissure is attached to a corresponding cell coupling member.

Example 174. The prosthetic valve of any example herein, particularly example 173, wherein each cell coupling member is sutures to the angled struts of the corresponding cell.

Example 175. The prosthetic valve of any example herein, particularly example 174, wherein each cell coupling member is made of a flexible fabric.

Example 176. The prosthetic valve of any example herein, particularly example 175, wherein the flexible fabric comprises a woven PET fabric.

Example 177. The prosthetic valve of any example herein, particularly any one of examples 173 to 176, wherein the tab stiff portions of each commissure are splayed in opposite directions along the circumferential direction to form a T-shape, such that a radially-outer surface formed by the splayed tab stiff portions contacts a radially-inner surface of the corresponding cell coupling member.

Example 178. A prosthetic valve, comprising:
a frame;
a non-uniform valvular structure coupled to the frame and configured to regulate flow through the prosthetic valve, the non-uniform valvular structure comprising:
a stiff portion extending between a valvular distal edge and an undulating boundary, the stiff portion comprising a stiff inflow region and a plurality of stiff post regions continuously extending from the stiff inflow region; and
a plurality of movable regions disposed between the undulating boundary and an outflow edge;
wherein the non-uniform valvular structure is formed of a unitary piece of material; and
wherein the stiff portion is stiffer than the movable regions.

Example 179. The prosthetic valve of any example herein, particularly example 178, wherein the non-uniform valvular structure is shape formed such that the stiff inflow region assumes an annular configuration, and the stiff post regions extend axially therefrom, in a free state of the non-uniform valvular structure prior to attachment to the frame.

Example 180. The prosthetic valve of any example herein, particularly example 178 or 179, wherein the non-uniform valvular structure is formed from natural tissue.

Example 181. The prosthetic valve of any example herein, particularly example 180, wherein the non-uniform valvular structure is formed from bovine pericardium.

Example 182. The prosthetic valve of any example herein, particularly any one of examples 178 to 181, wherein the ultimate tensile stress of the stiff portion is at least 2 times greater than the ultimate tensile stress of the movable regions.

Example 183. The prosthetic valve of any example herein, particularly any one of examples 178 to 182, wherein the stiff portion obtains a load at failure which is at least 3 times greater than the load at failure obtained by the movable regions.

Example 184. The prosthetic valve of any example herein, particularly any one of examples 178 to 183, wherein the plurality of movable regions comprises three movable regions.

Example 185. The prosthetic valve of any example herein, particularly any one of examples 178 to 184, wherein the movable regions are separated from each other by the stiff port regions.

Example 186. The prosthetic valve of any example herein, particularly any one of examples 178 to 185, wherein the movable regions are separated from each other by the stiff port regions.

Example 187. The prosthetic valve of any example herein, particularly any one of examples 178 to 186, wherein the stiff portions defines a minimal stiff portion height, and wherein the movable regions define a maximal movable region height which is greater than the minimal stiff portion height.

Example 188. The prosthetic valve of any example herein, particularly example 187, wherein the maximal movable region height is at least two times greater than the minimal stiff portion height.

Example 189. The prosthetic valve of any example herein, particularly example 187, wherein the maximal movable region height is at least three times greater than the minimal stiff portion height.

Example 190. The prosthetic valve of any example herein, particularly any one of examples 178 to 189, wherein the frame comprises a band.

Example 191. The prosthetic valve of any example herein, particularly example 190, wherein the stiff inflow region is attached to the band.

Example 192. The prosthetic valve of any example herein, particularly example 191, further comprising a sewing ring disposed around the band.

Example 193. The prosthetic valve of any example herein, particularly any one of examples 178 to 192, wherein the frame is devoid of vertically oriented commissure posts.

It is appreciated that certain features of the disclosure, which are, for clarity, described in the context of separate examples, may also be provided in combination in a single example. Conversely, various features of the disclosure, which are, for brevity, described in the context of a single example, may also be provided separately or in any suitable sub-combination or as suitable in any other described example of the disclosure. No feature described in the context of an example is to be considered an essential feature of that example, unless explicitly specified as such.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

1. A prosthetic valve comprising:
a frame movable between a radially compressed state and a radially expanded state;
a valvular structure coupled to the frame and comprising a plurality of non-uniform leaflets configured to regulate flow through the prosthetic valve, wherein each non-uniform leaflet comprises:
a movable body portion disposed between a free edge and an opposite cusp edge; and
at least one increased-stiffness portion;
wherein each non-uniform leaflet is formed from a unitary continuous piece of material; and
wherein the at least one increased-stiffness portion is stiffer than the movable body portion.

2. The prosthetic valve of claim 1, wherein the at least one increased-stiffness portion comprises an inflow portion extending between the cusp edge and an inflow portion proximal end.

3. The prosthetic valve of claim 2, wherein the frame comprises a plurality of intersecting struts, and wherein the inflow portion is coupled to some of the struts of the frame.

4. The prosthetic valve of claim 3, wherein the inflow portion defines an inflow portion width between the cusp edge and the inflow portion proximal end, wherein the struts to which the inflow portion is attached define a strut width between a strut inflow edge and a strut outflow edge, and wherein the inflow portion width is greater than the strut width.

5. The prosthetic valve of claim 4, wherein each non-uniform leaflet further comprises a pair of oppositely-directed tabs between the cusp edge and the free edge, and wherein the at least one increased-stiffness portion comprises tab stiff portions, each tab stiff portion extending from a tab outer edge of a corresponding tab to a tab portion inner boundary.

6. The prosthetic valve of claim 5, wherein each tab defines a tab length between the tab outer edge and an intersection of the tab with the cusp edge, wherein each tab stiff portion defines a stiff tab length between the tab outer edge and the tab portion inner boundary, and wherein the stiff tab length is greater than the tab length by an inner offsetting length.

7. The prosthetic valve of claim 6, wherein the tabs of adjacent non-uniform leaflets are joined together to form commissures attached, directly or indirectly, to the frame.

8. The prosthetic valve of claim 7, wherein the tab stiff portions are pre-shaped to assume a bent configuration in a free state prior to attachment commissure formation.

9. The prosthetic valve of claim 8, wherein the tabs stiff portions extend radially inward from the frame along a length which is equal to or greater than the offsetting length.

10. The prosthetic valve of claim 9, wherein the frame comprises commissure windows, each commissure window comprising a commissure window opening extending between window sidewalls, and wherein each commissure is attached to a corresponding commissure window.

11. The prosthetic valve of claim 10, wherein each of the tab stiff portion comprises a first section extending radially through a corresponding commissure window opening, and a second section folded sideways over a commissure window outer surface.

12. The prosthetic valve of claim 9, wherein the frame comprises commissure support posts, and wherein each commissure is attached to a corresponding commissure support post.

13. The prosthetic valve of claim 12, wherein each tab stiff portion comprises a first section extending radially from the tab portion inner boundary toward the commissure support post, a second section folded sideways over a support post inner surface, and a third section folded again to extend radially outward along at least a portion of a corresponding support post lateral side.

14. The prosthetic valve of claim 9, wherein the prosthetic valve further comprises cell coupling members, each cell coupling member extending across an opening of a cell formed by a plurality of interconnected angled struts of the frame, and wherein each commissure is attached to a corresponding cell coupling member.

15. The prosthetic valve of claim 9, wherein the tab stiff portions are sutured to interconnected angled struts of the frame defining corresponding cells of the frame.

16. A method of assembling a prosthetic valve, comprising the steps of:
providing a plurality of non-uniform leaflets, each non-uniform leaflet comprising a movable body portion disposed between a free edge and an opposite cusp edge, and at least one increased-stiffness portion; and
attaching the at least one increased-stiffness portion of each non-uniform leaflet to a frame movable between a radially compressed state and a radially expanded state;
wherein the at least one increased-stiffness portion is stiffer than the movable body portion.

17. The method of claim 16, wherein each non-uniform leaflet is formed from a unitary continuous piece of material.

18. The method of claim 16, wherein the at least one increased-stiffness portion comprises an inflow portion extending between the cusp edge and an inflow portion proximal end.

19. The method of claim 18, wherein attaching the at least one increased-stiffness portion of each non-uniform leaflet to the frame comprises attaching the inflow portion of each non-uniform leaflet to the frame.

20. The method of claim 19, wherein each non-uniform leaflet further comprises a pair of oppositely-directed tabs between the cusp edge and the free edge, and wherein the at least one increased-stiffness portion comprises tab stiff portions, each tab stiff portion extending from a tab outer edge of a corresponding tab to a tab portion inner boundary.
